(19) United States
(12) Patent Application Publication
(10) Pub. No.: US 2009/0156668 A1
(43) Pub. Date: Jun. 18, 2009

(54) Ginkgolide Compounds, Compositions, And Extracts, And Uses Thereof

(75) Inventors: Ottavio V. Vitolo, Cambridge, MA (US); Koji Nakanishi, New York, NY (US); Michael L. Shelanski, Brooklyn, NY (US); Sonja Krane, Del Mar, CA (US); Ottavio Arancio, New York, NY (US); Stanislav Jaracz, Trinec (CZ); Nina D. Berova, New York, NY (US)

Correspondence Address: WilmerHale/Columbia University, 399 PARK AVENUE, NEW YORK, NY, 10022, US

(73) Assignee: The Trustees Of Columbia University In The City Of New York, New York, NY (US)

(21) Appl. No.: 10/593,427

(22) PCT Filed: Mar. 21, 2005
(86) PCT No.: PCT/US2005/009417

§ 371 Date: Oct. 23, 2008

Related U.S. Application Data
(60) Provisional application No. 60/554,508, filed on Mar. 19, 2004.

(51) Int. Cl. A61K 31/365; C07D 493/22; A61K 51/00; C12N 5/02

(52) U.S. Cl. 514/462; 549/265; 424/1.65; 435/375

(57) ABSTRACT

The present invention relates to Ginkgolide derivatives, compositions and extracts comprising one or more Ginkgolides and/or derivatives thereof and methods of use of the compositions to treat neurological disorders and as imaging agents.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### 1. FIELD OF THE INVENTION

The present invention relates to Ginkgolide derivatives, compositions and extracts comprising one or more Ginkgolides and/or derivatives thereof and methods of use of the compositions to treat neurological disorders and as imaging agents.

### 2. BACKGROUND OF THE INVENTION

It is estimated that about 10% of the U.S. population is affected by a neurological disease or related disorder. The associated annual health care costs associated with neurological diseases in the U.S. alone are approximately six hundred billion dollars. Furthermore, it is estimated that particular types of neurological dysfunction, including Alzheimer's disease, memory loss and depression, are expected to grow acutely in frequency with the aging of the U.S. population.

One approach to treating various medical conditions, including neurological disorders, has been to look for naturally occurring, medicinally active substances from plants, marine life and other natural sources. Extracts from the Ginkgo biloba tree are currently among the best-selling herbal medications worldwide, with annual sales of approximately four billion dollars.

The G. biloba extract Egb 761, one of the first standardized preparations from the ginkgo tree, is extracted form the leaves of the Ginkgo biloba tree. This extract comprises contain 6% terpene trilactones (terpene trilactones) and flavonoids. The flavonoids are predominantly in the form of flavonol-O-glycosides, a combination of the phenolic aglycones quercetin, kaempferol, or isorhamnetin, with glucose or rhamnose or both at different positions of the flavonol moiety. The flavonoids may not penetrate the blood-brain barrier (BBB), whereas it appears that the lipophilic character of terpene trilactones renders these compounds permeable to the BBB, and therefore it appears that one or more of the terpene trilactones in G. biloba may be at least partially responsible for the observed neurological effects of G. biloba extracts. Several recent studies have measured the effects of the Egb 761 extract on various CNS disorders. These studies were performed using unrefined extracts, such as Egb 761, which contain ginkgolides A, B, C, and J, as well as bilobalide (BB) and a complex mixture of flavonoids and other components, and as such, do not address which of the known components of Egb 761, or other G. biloba extract, are efficacious neurologically, or the molecular basis for their action on the CNS. Further, although Egb 761 is potentially beneficial in relieving symptoms of neurodegenerative diseases and disorders, this and other G. biloba extract typically suffer from low efficacy, non-specificity and possible adverse effects when administered in vivo.

Accordingly, there is a need in the art for compounds and methods for treating CNS disorders with improved efficacy, specificity; and safety profiles. The present invention addresses that need.

### 3. SUMMARY OF THE INVENTION

In one aspect, the present invention provides Compounds having the Formula (I):

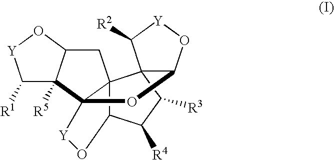

or a pharmaceutically acceptable salt thereof,
wherein:

each occurrence of Y is independently —CH₂— or —C(O)—;

R¹ and R³ are each independently —H or —C₁-C₆ alkyl;

R¹ is —H, —OH, —O—C₁-C₅ alkyl, —O—C₂-C₅ alkenyl, —O—C₂-C₅ alkynyl, —O—C(O)—C₁-C₅ alkyl, —O—C(O)-aryl, —O—CO—NH—C₁-C₅ alkyl, —O—SO₂—C₁-C₅ alkyl, or —O—SO₂-aryl;

R⁴ is —C₁-C₅ alkyl, —NH₂, -halo, —C₂-C₅ alkenyl, —C₂-C₅ alkynyl, —O—C₁-C₅ alkyl, —O—C₂-C₅ alkenyl, —O—C₂-C₅ alkynyl, —O—C(O)—C₁-C₅ alkyl, —O—C(O)-aryl, —O—CO—NH—C₁-C₅ alkyl, —O—SO₂—C₁-C₅ alkyl, or —O—SO₂-aryl; and

R⁵ is —H or —OH.

In another aspect, the present invention provides compositions consisting essentially of two or more compounds having the formula (II):

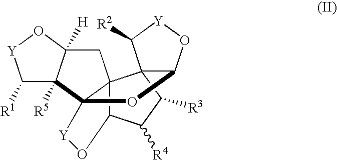

wherein:

each occurrence of Y is independently —CH₂— or —C(O)—;

R¹ and R³ and are each independently —H or —C₁-C₆ alkyl;

R¹ and R³ are each independently —H or —C₁-C₆ alkyl;

R² is —H, —H, —O—C₁-C₅ alkyl, —O—C₂-C₅ alkenyl, —O—C₂-C₅ alkynyl, —O—C(O)—C₁-C₅ alkyl, —O—C(O)-aryl, —O—CO—NH—C₁-C₅ alkyl, —O—SO₂—C₁-C₅ alkyl, or —O—SO₂-aryl;

R⁴ is —C₁-C₅ alkyl, —NH₂, -halo, —C₂-C₅ alkenyl, —C₂-C₅ alkynyl, —O—C₁-C₅ alkyl, —O—C₂-C₅ alkenyl, —O—C₂-C₅ alkynyl, —O—C(O)—C₁-C₅ alkyl, —O—C(O)-aryl, —O—CO—NH—C₁-C₅ alkyl, —O—SO₂—C₁-C₅ alkyl, or —O—SO₂-aryl; and

R⁵ is —H or —OH.

The invention also provides, compositions comprising Ginkgolide A and Ginkgolide J and derivatives thereof, wherein Ginkgolide A has the structure shown in formula 2 and Ginkgolide J has the structure shown in formula 3:

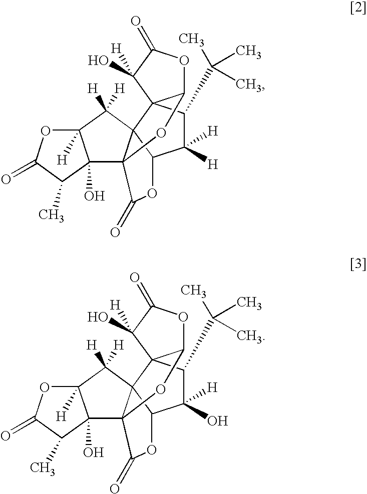

In one aspect, the invention provides a composition that consists essentially of Ginkgolide A and Ginkgolide J.

In another aspect, the invention provides compositions that consist essentially of Ginkgolide A, or a Ginkgolide A derivative, and Ginkgolide J, or a Ginkgolide J derivative, in combination with a pharmaceutical carrier.

The invention also provides methods to treat Alzheimer's disease comprising administering to a subject an effective amount of a composition consisting essentially of Ginkgolide A and Ginkgolide J.

The invention further provides methods to treat Alzheimer's disease comprising administering to a subject an effective amount of Ginkgolide J.

In another aspect, the invention provides methods for treating a neurological or neurodegenerative disease or disorder in a mammal by administering any of the aforementioned compositions. In a further aspect, the invention provides methods for treating a memory disorder in a mammal by administering any of the aforementioned compositions.

In still another aspect, the invention provides methods for improving or restoring memory in a mammal by administering any of the aforementioned compositions.

In a particularly useful aspect, the invention provides a method of treating depression in a mammal by administering any of the aforementioned compositions.

In another particularly useful aspect, the invention provides a method for protecting a neuron against neuronal cell death, and/or long term potentiation impairment, by beta amyloid protein. In this aspect, the method of the invention includes the step of contacting the neuron with Ginkgolide J.

In still another particularly useful aspect, the invention provides a method for protecting a neuron against neuronal cell death, and/or long term potentiation impairment, by beta amyloid protein. In this aspect, the method of the invention includes the step of contacting the neuron with a compound or composition of the invention.

In certain particularly useful embodiments of these methods, the neuron protected is contacted with a composition consisting essentially of Ginkgolide A and Ginkgolide J.

In one aspect, the invention provides a method for stimulating axonal outgrowth of a neuron by contacting the neuron with a composition of the invention.

In a further aspect, the invention provides a method for stimulating axonal outgrowth of a neuron by contacting the neuron with Ginkgolide A.

In a further aspect, the invention provides a method for stimulating axonal outgrowth of a neuron by contacting the neuron with Ginkgolide J.

In yet another aspect, the invention provides a method of treating a neurological or neurodegenerative disease or disorder in a mammal by administering an enriched Ginkgo biloba extract comprising at least about 60% terpene trilactones.

In another aspect, the invention provides a method of protecting a neuron against neuronal cell death, or long term potentiation impairment, by beta amyloid protein by contacting the neuron with an enriched Ginkgo biloba extract comprising at least about 60% terpene trilactones.

In a further aspect, the invention provides a method of identifying a receptor that binds a compound that protects against neuronal cell death or long term potentiation impairment by beta amyloid protein by administering a compound having the formula:

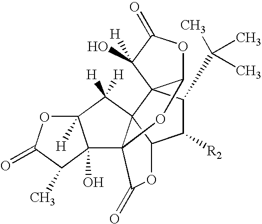

[5], where R₂ can be a photoactivatable moiety, a fluorescent moiety, or a radioactive moiety.

In still another aspect, invention provides a method of identifying a receptor that binds a compound that protects against neuronal cell death or long term potentiation impairment by beta amyloid protein by administering a compound having the formula:

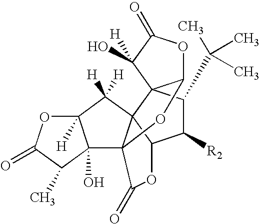

[4], where R₂ can be a photoactivatable moiety, a fluorescent moiety, or a radioactive moiety.

The present invention may be understood more fully by reference to the following detailed description and illustrative examples, which are intended to exemplify non-limiting embodiments of the invention.

### 4. BRIEF DESCRIPTION OF THE DRAWINGS

FIG. 1 is a graphical representation showing that a terpene trilactone 70% enriched preparation (P8A) reverses the inhibition of tetanus-induced neuronal long-term potentiation by amyloid-beta protein (βA).

FIG. 2A is a graphical representation showing that Ginkgolide A (GA) and Ginkgolide J (GJ) both reverse the inhibition by amyloid-beta protein (βA) of tetanus-induced neuronal long-term potentiation in the CA1 region of hippocampal slices.

FIG. 2B is a graphical representation showing Ginkgolide B (GB) and Ginkgolide C (GC), as well as bilobalide (BB) fail to reverse the inhibition of tetanus-induced neuronal long-term potentiation by amyloid-beta protein (βA).

FIG. 3 is a graphical representation showing the results of a neuroprotection assay which demonstrates that both a terpene trilactone enriched preparation (P8A) and Ginkgolide J alone protect hippocampal neurons from amyloid-beta protein (βA)-induced cell death.

### 5. DETAILED DESCRIPTION OF THE INVENTION

The patent and scientific literature referred to herein establishes knowledge that is available to those of skill in the art. The issued U.S. patents, allowed applications, published foreign applications, and scientific literature references, including GenBank database sequences, that are cited herein (including those in the Background of the Invention and all other sections) are hereby incorporated by reference to the same extent as if each was specifically and individually indicated to be incorporated by reference for all purposes.

### 5.1. General

The potentially potent and specific therapeutic benefits of one or more individual components remains untapped. Indeed, if only a subset of the terpene trilactones or other components are effective in treating or preventing neurological damage, then the potency of the treatment would be greatly increased by use of those individual component(s), which would avoid the potential for anti-therapeutic side-effects effects that might be caused by the remaining components.

The invention is based, in part, upon the finding that particular Ginkgolide species are potent molecular protectors against neurodegenerative processes including beta amyloid-induced neuronal cell death and impairment of long-term potentiation (LTP). For example, Ginkgolide A (GA) and Ginkgolide J (GJ) are potent in alleviating the inhibition of neuronal long-term potentiation caused by the neurodegenerative agent beta-amyloid protein (βA). Furthermore, Ginkgolide J is potent inhibitor of βA-induced neuronal cell death.

In general, the invention provides compositions for treating or preventing neurological diseases, disorders or conditions, and/or improving memory, as well as methods for their use. Certain of the ginkgolide compositions of the invention contain two or more ginkgolides or ginkgolide derivatives, particularly Ginkgolide A (GA) and/or a Ginkgolide A derivative, and Ginkgolide J and/or a Ginkgolide J derivative.

There are five ginkgolides (Ginkgolide A (GA), Ginkgolide B (GB), Ginkgolide C (GC), Ginkgolide J (GJ) and Ginkgolide M (GM)). The generalized structure of the ginkgolides is as follows in formula 1:

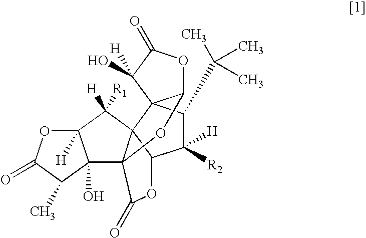

wherein, the structure at each of the variable “R” groups, R1, R2 and R3, varies uniquely in each ginkgolide species as follows: Ginkgolide A (R1=H, R2=H, R3=OH); Ginkgolide B (R1=OH, R2=H, R3=OH); Ginkgolide C(R1=OH, R2=OH, R3=OH); Ginkgolide J (R1=H, R2=OH, R3=OH); and Ginkgolide M (R1=OH, R2=OH, R3=H).

Accordingly, Ginkgolide A has the structure as follows in formula 2:

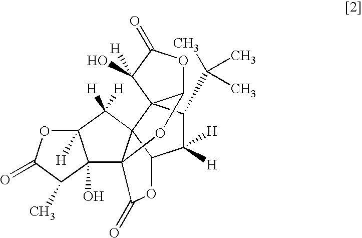

and Ginkgolide J has the structure as follows in formula 3:

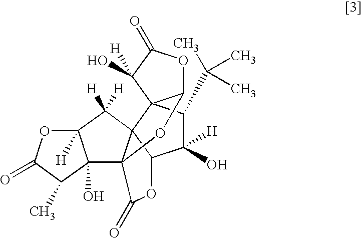

In one embodiment, the invention provides Ginkgolide J derivatives having formula 4:

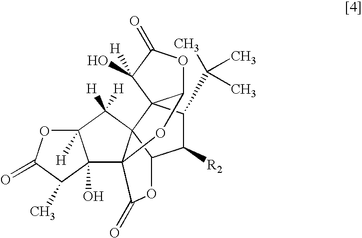

In another embodiment, the invention provides Ginkgolide A derivatives having formula 4:

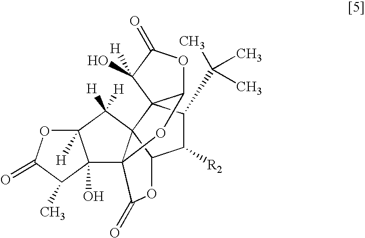

Furthermore, the invention provides an open-ringed monoacid form of Ginkgolides A and of Ginkgolide J, and derivatives thereof, obtained by the controlled hydrolysis of one of the lactone rings of the ginkgolide structure. The monoacid derivative of Ginkgolide A has the following structure (formula 6):

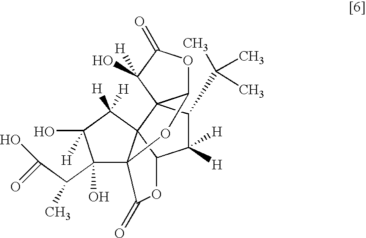

While the monoacid derivative of Ginkgolide J has the following structure (formula 7):

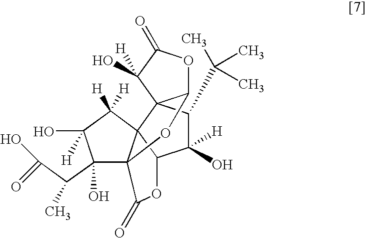

In each case, the “R²” of the ginkgolide derivative may be NH₂, F, Cl, Br, I, an unsubstituted or substituted straight or branched alkyl group having 1 to 5 carbon atoms, an alkenyl group having 1 to 5 carbon atoms, an alkynyl group having 1 to 5 carbon atoms, an O-alkyl having unsubstituted or substituted straight or branched alkyl group with 1 to 5 carbon atoms, an O-alkenyl group with 1 to 5 carbon atoms, an O-alkynyl group with 1 to 5 carbon atoms, an O—CO-alkyl ester having an unsubstituted or substituted straight or branched alkyl group with 1 to 5 carbon atoms, an O—CO-aryl ester that can be substituted or unsubstituted, an O—CO—NH-alkyl carbamate having an unsubstituted or substituted straight or branched alkyl group with 1 to 5 carbon atoms, an O—SO₂-alkyl sulfonate having an unsubstituted or substituted straight or branched alkyl group with 1 to 5 carbon atoms, or an O—SO₂-aryl sulfonate that can be substituted or unsubstituted.

### 5.2 Definitions and Abbreviations

The term “—C₁-C₅ alkyl” as used herein, refers to a straight chain or branched non-cyclic saturated hydrocarbon having from 1 to 5 carbon atoms, wherein one of the hydrocarbon's hydrogen atoms is replaced with a chemical bond. Representative straight chain —C₁-C₅ alkyls include -methyl, -ethyl, -n-propyl, -n-butyl and -n-pentyl. Representative branched —C₁-C₅ alkyls include -isopropyl, -sec-butyl, -isobutyl, -tert-butyl, -isopentyl, -neopentyl, 1-methylbutyl, 2-methylbutyl, 3-methylbutyl, 1,1-dimethylpropyl and 1,2-dimethylpropyl.

The term “—C₁-C₆ alkyl” as used herein, refers to a straight chain or branched non-cyclic saturated hydrocarbon having from 1 to 6 carbon atoms, wherein one of the hydrocarbon's hydrogen atoms is replaced with a chemical bond. Representative straight chain —C₁-C₆ alkyls include -methyl, -ethyl, -n-propyl, -n-butyl, -n-pentyl and -n-hexyl. Representative branched —C₁-C₆ alkyls include -isopropyl, -sec-butyl, -isobutyl, -tert-butyl, -isopentyl, -neopentyl, -neohexyl, 1-methylbutyl, 2-methylbutyl, 3-methylbutyl, 1,1-dimethylpropyl and 1,2-dimethylpropyl.

The term “—C₂-C₅ alkenyl” as used herein, refers to a straight chain or branched non-cyclic hydrocarbon having from 2 to 5 carbon atoms and including at least one carbon-carbon double bond, wherein one of the hydrocarbon's hydrogen atoms is replaced with a chemical bond. Representative —C₂-C₅ alkenyls include -vinyl, -allyl, -1-butenyl, -2-butenyl, -isobutylenyl, -1-pentenyl, -2-pentenyl, -3-methyl-1-butenyl, -2-methyl-2-butenyl, and -2,3-dimethyl-2-butenyl.

The term “—C₂-C₅ alkynyl” as used herein, refers to a straight chain or branched non-cyclic hydrocarbon having from 2 to 5 carbon atoms and including at least one carbon-carbon triple bond, wherein one of the hydrocarbon's hydrogen atoms is replaced with a chemical bond. Representative —C₂-C₅ alkynyls include -acetylenyl, -propynyl, -1-butynyl, -2-butynyl, -1-pentynyl, -2-pentynyl, -3-methyl-1-butynyl, and -4-pentynyl.

The term “aryl” as used herein, refers to a phenyl or naphthyl group.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

The term “β-amyloid protein” or “β-amyloid peptide,” refers to a glycoprotein associated with Alzheimer's disease and derived from a precursor, named A4, which is capable of being made in several forms through alternative splicing. The full-length human A4 protein is 770 amino acids long (86.85 kD) (see GenBank Accession Nos. P05067 and QRHUA4), while the β-amyloid protein is shorter carboxy-terminal fragment of the full-length (see GenBank Accession No. 1IYTA (Chain A, Solution Structure Of The Alzheimer's Disease Amyloid Beta-Peptide (1-42)) and, e.g., GenBank Accession No. AAA51726).

The term “antioxidant” refers to any substance, often an organic compound, that opposes oxidation or inhibits reactions brought about by dioxygen or peroxides. Usually the antioxidant is effective because it can itself be more easily oxidized than the substance protected. The term includes components that can trap free radicals, such as a-tocopherol (vitamin E), thereby breaking the chain reaction that normally leads to extensive biological damage. As used herein, the term “antioxidant” includes biological molecules that serve as natural free-radical scavengers intracellulary including ascorbate (vitamin C), glutathione, N-acetyl-L-cysteine and NAD⁺. “Antioxidant” further includes natural and synthetic compounds that protect labile compounds during storage or incubation (e.g., di-tert-butyl-p-cresol, and quinhydrone). The term includes plant flavonoids, such as the flavonol glycosides quercetin, kaempferol and isorhamnetin, as well as biflavones, such as amentoflavone, ailobetin, ginkgetin, isoginlgetin and sciadopirysin. Still other antioxidants included within the scope of the term are resveratrol, coenzyme Q, alpha-lipoic acid, and lycopene.

The term “detectable moiety” as used herein refers to a chemical group which can be attached to an organic molecule. Chemical groups useful as detectable moieties include, but are not limited to, a photoactivatable group, a fluorescent group, a radioactive group, or a group which can be detected using one or more medical imaging techniques including, but not limited to, x-ray, computed tomograpy (CT), magnetic resonance imagine (MRI), positron-emission tomography (PET), single photon emission computed tomograpy (SPECT), thermal imaging, fluoroscopic imaging, and ultrasound. In one embodiment, the detectable moiety is photoactivatable moiety, a fluorescent moiety, or a radioactive moiety.

An “effective amount,” when used in connection with a compound, composition, or extract of the invention, is an amount effective for treating or preventing Alzheimer's disease; treating or preventing a neurological or neurodegenerative disease or disorder; treating or preventing a memory disorder; improving memory; treating or preventing depression; protecting a neuron against neuronal cell death and/or long term potentiation impairment caused by beta amyloid protein; and stimulating axonal outgrowth of a neuron.

The term “flavonoid,” as used herein, includes any flavone, isoflavone, or neoflavone, or any of their derivatives, including especially flavone glycosides, biflavones, and bioflavonoids. Flavonoids include a large group of water-soluble phenolic derivatives, often brightly colored, and are typically found in the vacuoles of plant cells. They include the flavone glycosides, with the structure of their aglycon moieties based on the flavan skeleton, and are classified according to the oxidation state of their pyran ring (some 10-12 classes are well known).

The term “ginkgolide,” refers generally to a family of bioactive terpenes that were first isolated from the root bark and leaves of the Ginkgo biloba plant. The generalized structure of ginkgolide [1] varies at three carbon centers distinguished by a particular combination of side groups (R1, R2, and R3) unique to each form of Ginkgolide (i.e., A, B, C and J). The chemical identity of the side groups in each Ginkgolide is: Ginkgolide A (R1=H, R2=H, R3=OH); Ginkgolide B (R1=OH, R2=H, R3=OH); Ginkgolide C(R1=OH, R2=OH, R3=OH); Ginkgolide J (R1=H, R2=OH, R3=OH); and Ginkgolide M (R1=OH, R2=OH, R3=H).

The term “ginkgolide A derivative,” refers to ginkgolides having the structural formula [5], having a non-H R2 substituent present in the S chiral form, wherein the S chiral form is based upon an assignment of R2 as the highest priority substituent regardless of its actual priority over the other two substituents at this chiral center. In addition, the term “ginkgolide A derivative” is meant to include the monoacid derivative of Ginkgolide A having the structural formula [6].

The term “ginkgolide J derivative,” refers to ginkgolides having the structural formula [4], having a non-H R2 substituent present in the R chiral form, wherein the R chiral form is based upon an assignment of R2 as the highest priority substituent, regardless of its actual priority over the other two substituents at this chiral center. In addition, the term “ginkgolide J derivative” is meant to include the monoacid derivative of Ginkgolide J having the structural formula [7].

The term “glycoside,” refers to any of various kinds of molecules (including flavonoids, such as the flavonol glycosides or flavonol-O-glycoside), which have a sugar group covalently attached to the molecule. The corresponding molecule without the sugar (e.g., flavonol) is known as an “aglycone” when it is not bonded to a sugar molecule. The term “glycoside” as used herein, includes sugars forming an α- or β-glycosidic linkage. Representative examples of glycosides include, but are not limited to ribose, deoxynrbose, fructose, galactose, glucuronic acid and glucose.

The term “isolated,” used in reference to a compound, means that the compound is substantially free of other compounds, although other compounds may be present in trace amounts or in amounts insufficient to substantially alter the chemical or biological properties or the “isolated” compound. The term “isolated,” when applied to a composition or mixture of compounds, means that the composition is substantially free of compounds, other than those defining the composition.

The term “isomer” refers to any of two or more compounds that have identical molecular formulas but differ in the nature or arrangement of their atoms. The term “sterExtract of the Inventionsomers” refers to isomers that differ only in the three-dimensional arrangement of their atoms in space. Two sterExtract of the Inventionsomers typically differ in the arrangement of their atoms in space at one or more chiral centers. A chiral chemical compound, i.e., one that possesses chirality, is one that cannot be superimposed on its mirror image, either as a result of simple reflection or after rotation and reflection.

As used herein, the term “long-term potentiation” (or “LTP”) refers to any long-lasting enhancement of the effectiveness of synaptic transmission that follows certain types of conditioning stimulation. LTP is a form of long-term synaptic plasticity that is a critical cellular basis of learning and memory storage. For example, selective inhibition of NMDA receptor channels has been shown to block LTP, and to block spatial learning.

The term “substituted,” when referring to a chemical group that is part of a chemical compound, refers to the replacement of one or more hydrogen atoms of the chemical group with another atom or chemical group such as a halo atom, an amino group, an hydroxyl, or a carboxyl group.

A “subject” includes any mammal, e.g., a human, mouse, rat, guinea pig, dog, cat, horse, cow, pig, or non-human primate, such as a monkey, chimpanzee, or baboon.

### 5.3 The Compounds of Formula (I)

As stated above, the present invention encompasses Compounds of Formula (I):

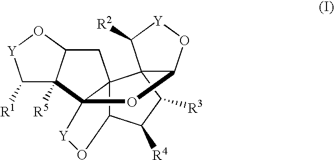

or a pharmaceutically acceptable salt thereof,
wherein Y, R¹, R², R³, R⁴ and R⁵ are defined above for the compounds of formula (I).

In one embodiment, each occurrence of Y is —CH₂—.

In another embodiment, each occurrence of Y is —C(O)—.

In one embodiment, R¹ is —H.

In another embodiment, R¹ is —C₁-C₆ alkyl.

In yet another embodiment, R¹ is methyl.

In one embodiment, R² is —H.

In another embodiment, R² is —OH.

In still another embodiment, R² is —O—C₁-C₅ alkyl, —O—C₂-C₅ alkenyl, or —O—C₂-C₅ alkynyl.

In yet another embodiment, R² is —O—C(O)—C₁-C₅ alkyl or —O—C(O)-aryl,

In another embodiment, R² is —O—CO—NH—C₁-C₅ alkyl.

In a further embodiment, R²—O—SO₂—C₁-C₅ alkyl, or —O—SO₂-aryl.

In one embodiment, R³ is —H.

In another embodiment, R³ is —C₁-C₆ alkyl.

In a further embodiment, R³ is tert-butyl.

In one embodiment, R⁴ is —OH.

In another embodiment, R⁴ is —C₁-C₅ alkyl, —C₂-C₅ alkenyl or —C₂-C₅ alkynyl.

In still another embodiment, R⁴ is —NH₂.

In yet another embodiment, R⁴ is -halo.

In a further embodiment, R⁴ is —O—C₁-C₅ alkyl, —O—C₂-C₅ alkenyl or —O—C₂-C₅ alkynyl.

In yet another embodiment, R⁴ is —O—C(O)—C₁-C₅ alkyl or —O—C(O)-aryl,

In another embodiment, R⁴ is —O—CO—NH—C₁-C₅ alkyl.

In a further embodiment, R⁴—O—SO₂—C₁-C₅ alkyl, or, —O—SO₂-aryl.

In one embodiment, R⁵ is —H.

In another embodiment, R⁵ is —OH.

In one embodiment, the compounds of formula (I) have the formula (Ia):

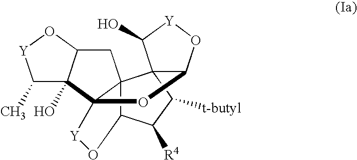

Wherein R⁴ is —H, —C₁-C₅ alkyl, —OH, —NH₂, -halo, —C₂-C₅ alkenyl, —C₂-C₅ alkynyl, —O—C₁-C₅ alkyl, —O—C₂-C₅ alkenyl, —O—C₂-C₅ alkynyl, —O—C(O)—C₁-C₅ alkyl, —O—C(O)-aryl, —O—CO—NH—C₁-C₅ alkyl, —O—SO₂—C₁-C₅ alkyl, or —O—SO₂-aryl.

In one embodiment, the Compounds of formula (I) are useful for treating or preventing the following conditions in a subject a neurological or neurodegenerative disease or disorder; a memory disorder, or depression.

In another embodiment, Compounds of formula (I) are useful for: protecting a neuron against neuronal cell death, and/or long term potentiation impairment by beta amyloid protein; and stimulating axonal outgrowth of a neuron.

The compounds of formula (Ia) can be made by converting the 7-OH group of ginkgolide J to a good leaving group, such as —O-triflyl, —O-mesyl, —O-tosyl, —O-brosyl, —Cl, —Br, or —I using methods well known to one of skill in the art of organic chemistry. The leaving group can then be displaced by a nucleophile corresponding to any of the R⁴ groups as defined above for the Compounds of formula (I).

### 5.4 Compositions Comprising Compounds of Formula (II)

As stated above, the present invention encompasses compositions consisting essentially of two or more structurally distinct compounds of Formula (II):

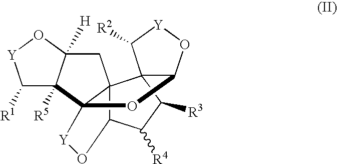

wherein Y, R¹, R², R³, R⁴ and R⁵ are defined above for the compounds of formula (II).

In one embodiment, the composition comprises at least one compound of formula (II), wherein each occurrence of Y is —CH₂—.

In another embodiment, the composition comprises at least one compound of formula (II), wherein each occurrence of Y is —C(O)—.

In one embodiment, the composition comprises at least one compound of formula (II), wherein R¹ is —H.

In another embodiment, the composition comprises at least one compound of formula (II), wherein R¹ is —C₁-C₆ alkyl.

In yet another embodiment, the composition comprises at least one compound of formula (II), wherein R¹ is methyl.

In one embodiment, the composition comprises at least one compound of formula (II), wherein R² is —H.

In another embodiment, the composition comprises at least one compound of formula (II), wherein R² is —OH.

In still another embodiment, the composition comprises at least one compound of formula (II), wherein R² is —O—C₁-C₅ alkyl, —O—C₂-C₅ alkenyl, or —O—C₂-C₅ alkynyl.

In yet another embodiment, the composition comprises at least one compound of formula (II), wherein R² is —O—C(O)—C₁-C₅ alkyl or —O—C(O)-aryl,

In another embodiment, the composition comprises at least one compound of formula (II), wherein R² is —O—CO—NH—C₁-C₅ alkyl.

In a further embodiment, the composition comprises at least one compound of formula (II), wherein R²—O—SO₂—C₁-C₅ alkyl, or —O—SO₂-aryl.

In one embodiment, the composition comprises at least one compound of formula (II), wherein R³ is —H.

In another embodiment, the composition comprises at least one compound of formula (II), wherein R³ is —C₁-C₆ alkyl.

In a further embodiment, the composition comprises at least one compound of formula (II), wherein R³ is tert-butyl.

In one embodiment, the composition comprises at least one compound of formula (II), wherein R⁴ is —H.

In another embodiment, the composition comprises at least one compound of formula (II), wherein R⁴ is —C₁-C₅ alkyl, —C₂-C₅ alkenyl or —C₂-C₅ alkynyl.

In still another embodiment, the composition comprises at least one compound of formula (II), wherein R⁴ is —NH₂.

In yet another embodiment, the composition comprises at least one compound of formula (II), wherein R⁴ is -halo.

In a further embodiment, the composition comprises at least one compound of formula (II), wherein R⁴ is —O—C₁-C₅ alkyl, —O—C₂-C₅ alkenyl or —O—C₂-C₅ alkynyl.

In yet another embodiment, the composition comprises at least one compound of formula (II), wherein R⁴ is —O—C(O)—C₁-C₅ alkyl or —O—C(O)-aryl,

In another embodiment, the composition comprises at least one compound of formula (II), wherein R⁴ is —O—CO—NH—C₁-C₅ alkyl.

In a further embodiment, the composition comprises at least one compound of formula (II), wherein R⁴—O—SO₂—C₁-C₅ alkyl, or —O—SO₂-aryl.

In one embodiment, the composition comprises at least one compound of formula (ED, wherein R⁵ is —H.

In another embodiment, the composition comprises at least one compound of formula (II), wherein R⁵ is —OH.

In one embodiment, the compound of formula (II) has the formula:

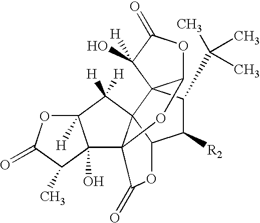

wherein R₂ is —H, —C₁-C₅ alkyl, —OH, —NH₂, -halo, —C₂-C₅ alkenyl, —C₂-C₅ alkynyl, —O—C₁-C₅ alkyl, —O—C₂-C₅ alkenyl, —O—C₂-C₅ alkynyl, —O—C(O)—C₁-C₅ alkyl, —O—C(O)-aryl, —O—CO—NH—C₁-C₅ alkyl, —O—SO₂—C₁-C₅ alkyl, or —O—SO₂-aryl.

In another embodiment, the compound of formula (II) has the formula:

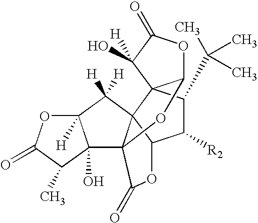

wherein R₂ is —H, —C₁-C₅ alkyl, —OH, —NH₂, -halo, —C₂-C₅ alkenyl, —C₂-C₅ alkynyl, —O—C₁-C₅ alkyl, —O—C₂-C₅ alkenyl, —O—C₂-C₅ alkynyl, —O—C(O)—C₁-C₅ alkyl, —O—C(O)-aryl, —O—CO—NH—C₁-C₅ alkyl, —O—SO₂—C₁-C₅ alkyl, or —O—SO₂-aryl.

Illustrative examples of the compounds of Formula (II) include Ginkgolide A and Ginkgolide J.

In one embodiment, the compositions consisting essentially of two or more compounds of formula (II) are useful for treating or preventing the following conditions in a subject: a neurological or neurodegenerative disease or disorder; a memory disorder; or depression.

In another embodiment, the compositions consisting essentially of two or more compounds of formula (II) are also useful for: protecting a neuron against neuronal cell death, and/or long term potentiation impairment by beta amyloid protein; and stimulating axonal outgrowth of a neuron.

In one embodiment, the compositions consisting essentially of two or more compounds of formula (II) may further contain one or more of the following compounds:

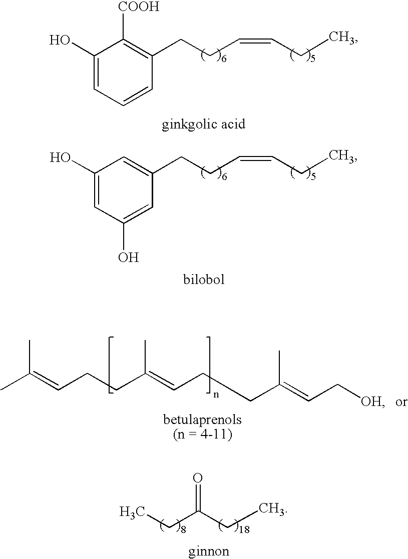

### 5.5 Ginkgolides A and J and Related Derivatives

The invention provides isolated ginkgolides, particularly Ginkgolides A and J (GA and GJ), as well as derivatives of these ginkgolides. Ginkgolides A and J may be obtained from a natural source, such as an unenriched Ginkgo biloba extract (such as Egb761), or an enriched terpene trilactone (TTL) extract (see e.g., U.S. Pat. No. 6,590,109), or may be chemically synthesized (e.g., by de novo chemical synthesis as has been described by E. J. Corey (see Corey and Cheng (1995) The Logic of Chemical Synthesis, John Wiley & Sons, New York, pp. 89-91, 221-226). The ginkgolides and ginkgolide derivatives of the invention may be obtained by chemical synthesis using such known chemical methods, and/or routine modifications of such methods.

Where the ginkgolides are derived from a natural source, such as Ginkgo biloba plant parts, they will generally be most efficiently isolated in a relatively pure state by first isolating an enriched mixture of plant terpene trilactones. The first step in obtaining such an enriched TTL preparation may be extraction from the leaves. In most cases, various water-containing solvent systems such as water/acetone or water/methanol are used. Apolar constituents are excluded, whereas all of the terpene trilactones are collected, including GB, which, like the other ginkgolides, is only scarcely soluble in water. Basic extractions should be avoided owing to the instability of BB (bilobalide) in solutions with pH>7. Recently, an improved extraction of terpene trilactones that takes advantage of their stability under a variety of conditions, such as oxidation and heat, was developed (see Lichtblau et al. (2002) J. Nat. Prod. 65: 1501). During this process, the leaves are boiled for 1 hour in dilute hydrogen peroxide, followed by extraction with ethyl acetate to generate an off-white powder with a TTL content of 60-70%. The treatment with hydrogen peroxide removes the constituents that lead to extensive emulsions in the extraction steps, thus greatly shortening the process.

Methods useful for preparing enriched TTL extracts have been described (see U.S. Pat. No. 6,590,109). In brief, the method involves isolating and concentrating terpene trilactones from pharmaceutical powder produced by extraction of the leaves of Ginkgo biloba, for example Egb761, and involves the following procedures: (A) The dried leaves or the pharmaceutical powder are suspended in water, or in an aqueous solution containing hydrogen peroxide at a concentration range of 0.1-30%, or in an aqueous solution containing hydrogen peroxide at a concentration range of 0.1-30% and additional 0.1-15% percent of mineral acids like acetic, hydrochloric, nitric, phosphoric or sulfuric acid; (B) the resulting suspension is stirred only, or stirred and heated, or stirred and boiled for a period of time between 5 min to 5 hours; (C) if necessary, the pH is adjusted after this treatment to a range of pH 4-6 with alkali hydroxide aqueous solutions such as sodium hydroxide or potassium hydroxide; (D) the suspension of (B) or (C) is extracted with lower acetates, lower ketones, lower ether, lower alcohols or benzenes, by separating the organic layer from the aqueous solution; (E) the resulting organic layer is washed with an aqueous based solution, such as ammonium chloride, sodium carbonate, sodium bicarbonate, potassium carbonate or with alkali hydroxide aqueous solutions such as sodium hydroxide or potassium hydroxide, or with an aqueous solutions such as sodium thiosulfate, sodium sulfite and sodium hydrosulfide or destroy the excess of hydrogen peroxide with metal dust or on metal based catalysts, or with both such solutions; the organic layer is washed water and/or with an aqueous solution of sodium chloride; (G) the remaining organic layer is separated, dehydrated and dried, so as to remove solvent and obtain an extract which contains more then 50% terpene trilactones; and (H) the extract from step (G) is recrystallized with a mixture of water/methanol or any other acceptable solvent system to obtain the ginkgolides in high purity.

Next, to obtain individual isolated ginkgolide species such as GA and/or GJ, the individual ginkgolides and bilobalide (BB) are separated. The terpene trilactones can be isolated by further processing, for example by reversed phase chromatography, to reduce any unwanted ginkgolic acids. For example, chromatography can be performed with silica gel.

Bilobalide is relatively easily separated from the ginkgolides by using standard column chromatography. The separation of the individual ginkgolides requires further methodology. The ginkgolides differ only by one or two hydroxyl groups, but in some cases these hydroxyl groups are involved in hydrogen bonding, and therefore do not significantly alter the overall polarity of the molecule. Accordingly, a simple improvement in the separation has been described by van Beek and Lelyveld, in which the hydrogen bonds are disrupted by using silica gel impregnated with sodium acetate. This technique is coupled with preparative-scale medium-pressure liquid chromatography (MPLC) (van Beek et al. (1997) J. Nat. Prod. 60: 735) and TLC detection of terpene trilactones (van Beek et al. (1993) Phytochem. Anal. 4: 109). In the purification step, the labile nature of BB should be considered, as it degrades on alumina columns (van Beek et al. (1996) Phytochem. Anal. 7:185). Quantitative ¹H NMR spectroscopic analysis was used as a convenient method to determine the amount of terpene trilactones in various preparations; (Choi et al. (2003) Chem. Pharm. Bull. 51: 158) since the signals for the 12-H of terpene trilactones (Rounestand et al. (1989) Tetrahedron 45: 1975) are distinct and well-separated, integration intensities of these signals are then compared to that of maleic acid (MA) (van Beek et al. (1993) Phytochem. Anal. 4:261).

Another useful method, albeit most practical for rapid analytical, rather than preparative, procedures is a method using HPLC on a Lichrosorb C18 (10 um) analytical column with methanol-water-orthophosphoric acid (25:75:0.1, v/v) as eluent and refractive indes (RI) detection (see Wang et al. (2000) Chin. J. Chrom. 18: 394-97).

terpene trilactones lack common chromophores and therefore UV detection is not ideal. Accordingly, other detection methods, such as refractive index (RI), evaporation light-scattering detection (ELSC), and MS have been utilized. The first reported separation and quantification of terpene trilactones, however, involved conventional HPLC/V detection (see Teng et al. (1988) J. Chromatogr. 267: 431). RI detection has also been described (van Beek et al. (1991) J. Chromatogr. 543: 375). Although UV detection often used because it demonstrates better sensitivity that RI detection, the selectivity is superior when using RI. Several methods have been described using liquid chromatography/mass spectrophotometry (LC/MS) to separate and quantify TTL content in various G. biloba extracts, as well as in plasma after intake of G. biloba extract. The major difference in the detection systems lies in the MS procedures used, which could be electrospray ionization (ESI) (Mauri et al. (1999) J. Mass Spectrom. 34: 1361) or atmospheric-pressure chemical ionization (APCI) (Mauri et al. (2001) Rapid Commun. Mass Spectrom. 15: 929; Jensen et al. (2002) Phytochem. Anal. 13: 31). Furthermore, LC/MS (ESI) has been used for analysis of commercial G. biloba products in which large variations in the composition and concentration of terpene trilactones were observed (Li et al. (2002) Analyst 127: 641). In addition, as an alternative to MS, ELSD was also successfully applied to quantify terpene trilactones in G. biloba extracts (Camponovo et al. (1995) Phytochem. Anal. 6: 141; Ganzera et al. (2001) Chem. Pharm. Bull. 49: 1170).

In addition to purification from a natural source, the ginkgolides and related compounds of the invention may be chemically synthesized. Corey and co-workers have described the total syntheses of both GA (Corey and Ghosh (1988) Tetrahedron Lett. 29: 3205) and GB (Corey et al. (1988) J. Arm. Chem. Soc. 110: 649). The classic total synthesis of GB has been extensively reviewed (Nicolaou et al. (1996) Classics in Total Synthesis VCH, Weinheim p. 451) and only the detailed methodology is well known in the art. Briefly, the key transformations include the following.

A spirocyclic ring system composed of rings B and C of GB is constructed from 2-(2,2-dimethoxyethyl)-cyclopent-2-enone by a 1,4-addition of a tert-butyl cuprate reagent, followed by a Mukaiyama condensation after treatment with 1,3,5-trioxane and titanium chloride to give an intermediate using the tert-butyl moiety as a directing group for the ring closure. A sequence of steps involving a palladium-mediated Sonogashira coupling, an intramolecular ketene-olefin [2+2]cycloaddition, and a Baeyer-Villiger oxidation furnished an intermediate, which contains four of the six rings in GB. For the formation of the tetrahydrofuran moiety, ring D, the reactivity of ring C was modified by introduction of dithiane, and intramolecular etherification provided this intermediate in three steps. Oxidation of ring A and elimination in ring C furnished an intermediate, which was selectively oxidized to provide an epoxyketone in ring A. This was followed by an intermolecular aldol condensation and lactonization with concomitant opening of the oxirane to give an intermediate with all six rings of GB in place. The total synthesis was completed by dihydroxylation of the double bond in ring C and oxidation to GB. Later, Corey and co-workers suggested an enantioselective route to GB by synthesis of a key intermediate (Corey et al. (1988) Tetrahedron Lett. 29: 3201) and various ginkgolide derivatives were also synthesized (Corey et al. (1990) Tetrahedron Lett. 31: 3995; Corey et al. (1991) Tetrahedron Lett. 32: 4623).

Another method for synthesis of GB has been reported by Crimmins and coworkers (Crimmins et al. (1989) Tetrahedron Lett. 30:5997). This method utilizes an intramolecular cycloaddition reaction that efficiently generated the multiple-ring skeleton of ginkgolides. The synthesis started from a furan enone which is very similar to an intermediate created in the synthesis of bilobalide. This furan enone undergoes intramolecular photocycloaddition upon irradiation at >350 nm to give a polycyclic structure analogous to rings A, B, and C of GB, as a single diastereomer. Inversion of the MOM-protected (methoxymethyl-protected) alcohol and formation of a bridging lactone, is followed by ring expansion of the cyclobutane into a tetrahydrofuran system in six steps to give the desired structure, which is comparable to the analogous Corey intermediate, but lacks crucial functional groups and the tert-butyl group (Crimmins et al. (1989) Tetrahedron Lett. 30:5997). In yet another method for the total synthesis of GB, a photocycloaddition is used as a key transformation (Crimmins et al. (1999) J. Am. Chem. Soc. 121: 10249).

Methods for synthesizing ginkgolide derivatives, particularly Ginkgolide A and Ginkgolide J derivatives, have been described and/or may be elucidated using routine skill. Numerous synthetic modifications of the ginkgolide parent compounds have been carried out. Methods for obtaining synthetic derivatives of the ginkgolides have been described in, for example, U.S. Pat. Nos. 6,693,091 and 5,541,183.

For example, two approaches have been developed for converting GC into GB, the most potent ginkgolide PAF receptor antagonist. First, Weinges and Schick described a four-step procedure in which the 1-OH group of GC was protected as a tert-butyldiphenylsilyl (TBDPS) ether prior to treatment with phenyl chlorothionoformate to give an intermediate. This compound was then treated with Bu₃SnH and azobisisobutyronitrile (AIBN) in a Barton-McCombie alcohol deoxygenation, and GB was liberated by removal of the silyl protecting group (Weinges et al. (1991) Liebigs Ann. Chem. 81). Similarly, Corey et al. protected GC as a benzyloxymethyl (BOM) ether at the hydroxyl at position 10, and followed a similar path to form GB (see Corey et al. (1992) Tetrahedron Lett. 33:6955). A very convenient, two-step procedure was described by Teng (Teng (1995) GB 2288599 and Chem. Abstr. 124: 117659) in which GC was treated with triflic anhydride to yield exclusively 7-O-triflate-GC, which was reduced with Bu₄NBH₄ to give GB.

The different reactivities of the 1-, 7-, and 10-OH groups of the ginkgolides are noteworthy: A bulky silyl group reacts preferentially at 1-OH, whereas all alkylation reagents react at 10-OH and triflic anhydride reacts exclusively at 7-OH of GC under strongly acidic conditions (Jaracz et al. (2002) J. Org. Chem. 467:4623). Generally, 1- and 10-OH are the most reactive OH groups owing to the hydrogen bonding between the two and hence the relative ease of formation of a delocalized alkoxy anion (Corey et al. (1992) Tetrahedron Lett. 33:6955; Jaracz et al. (2002) J. Org. Chem. 67:4623). The increased reactivity of 7-OH towards sulfur nucleophiles may be due to the soft-atom nature of sulfur. Accordingly, sulfur nucleophiles are particularly useful in the synthesis of certain Ginkgolide J derivatives.

During the structural studies of ginkgolides and bilobalide, several analogues were synthesized. For example, acetylated ginkgolides and bilobalide (Nakanishi et al. (1971) J. Am. Chem. Soc. 93:3544; and Weinges et al. (1972) Justus Liebigs Ann. Chem. 759, 158) were described, as well as the iso-derivatives, which stem from a translactonization of ring E in ginkgolides. Recently, an X-ray crystal structure of the iso-derivative 1,6,10-triacetate-isoGC was obtained, and a mechanism for the formation of iso-derivatives that includes opening of ring E, stabilization of the intermediate by hydrogen bonding to 3-OH, and capture by acetic anhydride was suggested (Jaracz et al. (2002) J. Org. Chem. 67:4623). A similar translactonization was described by Weinges et al. upon acetylation of BB and during the preparation of various acyl derivatives of BB (Weinges et al. (1987) Liebigs Ann. Chem. 1079).

Weinges et al. has carried out numerous synthetic studies of ginkgolides and bilobalide (Weinges et al. (1986) Liebigs Ann. Chem. 1057) (Weinges et al. (1993) Liebigs Ann. Chem. 1023) (Weinges et al. (1993) Liebigs Ann. Chem. 287) (Weinges et al. (1997) Ann. Chem. 1607) (Weinges et al. (1997) Ann. Chem. 1755) (Weinges et al. (1997) Ann. Chem. 991) (see also, the preparation of radiolabeled analogues of ginkgolides, in this case [¹⁴C] GA (Weinges et al. (2000) Tetrahedron 56:3173)).

When ginkgolides are tested in pharmacological assays it may be necessary to make a concentrated stock solution of the compound in dimethyl sulfoxide (DMSO), owing to their low solubility in water. DMSO may cause problems in certain assay systems, (Maclennan et al. (1996) Neurosci. Res. 26:395) although such effects are not uniformly observed (Strømgaard et al. (2002) J. Med. Chem. 45:4038). To increase the water solubility of ginkgolides, Weber and Vasella synthesized glycosylated ginkgolides analogues (Weber et al. (1997) Helv. Chim. Acta 80:2352) (Vasella et al. (1998) (SCRAS) U.S. Pat. No. 6,143,725 Chem. Abstr. 130:38639). Glycosidation was carried out by reaction of ginkgolides with a glycosylated analogues. Intra- and intermolecular bonds of these analogues, as well as the parent ginkgolides, were studied by ¹H NMR spectroscopic analysis (Bernet et al. (2000) Helv. Chim. Acta 83:995).

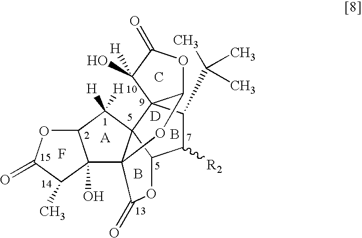

Ginkgolides contain three lactones (designated rings C, E, and F) (see structure [8] above, which shows the ring nomenclature scheme for the generalized GA/GJ ginkgolide structure as well as the carbon atom numbering scheme used in ginkgolide nomenclature) and hence their structures in solution are highly dependent on the pH value of the media in which they are dissolved. Zekri et al. determined the ionization constants of ginkgolides by ¹H NMR titrations (Zekri et al. (1996) Anal. Chem. 68:2598), which showed that the lactones start to open at around pH 7. At pH 8, the predominant species (≈50%) is the form in which only ring F is open, ring E is open to a lesser extent (≈20%). An increase in the pH value to 10 opens both rings E and F (≈90%). Only at pH 13 is the species with all the lactones open present (≈40%), although about 60% is still in the form with only rings E and F open. Chemical schemes for the opening and/or removal of one or more of these ginkgolide lactone rings have been described (Hu et al. (2001) J. Asian Nat. Prod. Res. 3: 219-22; Corey et al. (1989) Tetrahedron Lett. 30: 6959-62; and Weinges et al. (1997) Liebigs Ann. Chem. 1997: 991-3).

For example, Hu et al. ((2001) J. Asian Nat. Prod. Res. 3: 219-22) demonstrate the synthesis and biological activity of amide analogues of GA. Briefly, GA is treated with Jones reagent (an acidic aqueous solution containing chromic acid), which is added to an acetone solution of the GA to be oxidized. This treatment yields 10-oxoginkgolide, which is in equilibrium with its ring-C-open analogue, and a novel heptacyclic ginkgolide analogue, the formation of which is formed as a result of the formation of radicals from the t-butyl methyls of 10-oxoginkgolide A under sunlight irradiation. Reaction of 10-oxoginkgolide A with benzylamine in the THF solution and refluxing for 2 hours resulted in the formation of an amide derivative of the C-ring opened form of GA in which the PhCH₂NH— group was connected to C₁₁ to form benzyl 11-ginkamide. A similar strategy was employed to synthesize other amide derivative of ginkgolide A from 10-oxoginkgolide A and an appropriate amine using the same method (i.e., in addition to the C₁₁—NH—CH₂Ph amide, a C₁₁—NH-Ph amide (phenyl 11-ginkamide), a C₁₁—NH—CH₃ amide (methyl 11-ginkgamide), a C₁₁—NH₂ amide (11-ginkgamide), a C₁₁—NH—OH (hydroxyl 11-ginkgamide), and a morpholino-11-ginkamide ginkgolide derivative were synthesized. Notably, these ring-C-open forms of GA (including amide and monoacid forms) retained substantial amounts of biological activity in a PAF-induced (platelet activating factor-induced) platelet aggregation assay IC50 (uM) (Hu et al. (2001) J. Asian Nat. Prod. Res. 3: 219-22). Analogous strategies may be employed to obtain the C-ring opened, monoacid and analogous amide forms of GJ.

“Simple Analogs” of ginkgolide B that are missing the “F” ring structure have been synthesized, and shown to retain high levels of biological activity as antagonists of platelet activating factor (Corey and Gavai (1989) Tetrahedron Lett. 30: 6959-62). Briefly, the synthesis of these “simple analogs” of ginkgolide molecules with anti-PAF activity was the tetracyclic lactone, a key intermediate in the total synthesis of ginkgolide B described previously. (Corey et al. (1988) J. Am. Chem. 110:649; and Corey et al. (1988) Tetrahedron Letters 29:3201). The racemic form of the tetracyclic lactone was employed since a sizeable quantity of this compound was available from earlier work and all of the analogs of the tetracyclic lactone described here were obtained as racemates. Initial studies of the anti-PAF activity of early-stage, tetracyclic synthetic intermediates lacking the oxygen bridge between C(4) and C(12) had indicated very low biological potency (IC₅₀>100 μM). In contrast, the lactone subunit attached to C(2) and C(3) of GB is not essential to biological activity, as indicated by the information which follows.

The tetracyclic lactone was transformed in five steps via to the chlorohydrin bis-lactone, by the following chemical synthetic sequence: (1) stereospecific α-epoxidation of the C(1)-C(2) olefinic linkage (m-chloroperoxybenzoic acid in CH₂Cl₂, pH 8 aqueous phosphate buffer at 23° C., 92%); (2) oxirane ring opening to form a chlorohydrin intermediate (3 equiv of BCl₃ and 4 equiv. of benzyltriethylammonium chloride in CH₂Cl₂ at −45° C. to 23° C., 79%); (3) elimination of methanol to convert the methyl acetal intermediate to the corresponding dihydrofuran (heating with 5 equiv. of each pyridinium tosylate and pyridine in chlorobenzene at 135° C. for 16 h, 83%); (4) dihydroxylation of the C(10)-C(11) olefinic linkage (osmium tetroxide-pyridine, 55° C. for 36 h, 69%); and (5) oxidation of lactol to lactone (I₂, aqueous MeOH containing CaCO₃ at 23° for 0.5 h, 82%). The resulting bis lactone diol was also converted to the corresponding bis methoxymethyl (MOM) ether, (excess CH₂(OMe)₂, P₂O₅ in ClCH₂CH₂Cl at 23° C., 69%). The anti-PAF activity of this intermediate was measured to be IC₅₀=0.3 uM as compared to IC₅₀=(0.6 uM for ginkgolide B as a control. The diol intermediate (IC₅₀=1.1 uM) was somewhat less active than the bis MOM derivative, indicating that free hydroxyl groups are not necessary for anti-PAF biological activity of ginkgolides. Assuming that only one enantiomer of the MOM derivative is active, it follows that chiral MOM derivative is about four times more potent as an anti-PAF agent than ginkgolide B.

The 2-bromo analogs of the diol intermediate and the bis MOM derivative were synthesized from the tetracyclic lactone in a parallel fashion, and their anti-PAF IC₅₀ values were determined as 14 μM and 0.6 μM, respectively.

Two 1α, 2β-dichloro derivatives (with either C(10)-OH or C(10)-OCH₂OCH₃), were also synthesized individually from the tetracyclic lactone by a sequence consisting of 1α,2β -dichlorination (chlorine and benzyltriethylammonium chloride in CH₂Cl₂-CF₃CH₂OH at 0° C., 65%) and then functional group modification at C(10) and C(11) as described above for the synthesis of the diol intermediate and the bis MOM derivative. The anti-PAF IC₅₀ values of the two 1α, 2β-dichloro derivatives (having C(10)-OH and —OCH₂OCH₃), were determined to be 0.4 μM and 0.2 μM, respectively. Thus, the active dichloro derivative enantiomer having C(10)-OCH₂OCH₃ is expected to be ca. six times as active as ginkgolide B with IC₅₀=0.1 μM. Since this dichloro entaniomer is considerably less polar than ginkgolide B, it is expected to be much better absorbed after p.o. administration, and possibly more efficacious.

The C(10) epimer of 1α, 2β-dichloro derivatives (with C(10)-OH) was synthesized by oxidation to the corresponding α-keto lactone (Jones' reagent, acetone-water, 23° C. for 1 h) and subsequent reduction using excess aluminum amalgam in 20:1 THF—H₂O at 23° C. for 2 h. The anti-PAF IC₅₀ value for the C(10)-epimer of this dichloro enantiomer was found to be 1.3 μM. The isomer of this dichloride having an oxygen bridge between C(4) and C(8), was synthesized from the related C(1)-C(2)-olefin (Corey et al. (1988) J. Am. Chem. 110:649) and found to be considerably less active, IC₅₀=38 μM.

A carbonyl function at C(11) is beneficial for anti-PAF activity, but not essential; thus, the IC₅₀ for the tetracyclic lactone was 120 μM as compared to 80 μM for the corresponding structure having a carbonyl group at C(11).

The effect of substituents at C(10) was evaluated for the series several related derivatives, having no substituents at C(1) and C(2). The following IC₅₀ values were exemplary for those derivatives: 76 μM; 13 μM; 9.4 μM; 13 μM; 11 μM; and 21 μM. The α-keto lactone obtained by oxidation of the 10-hydroxyl of some of these related derivatives, which showed an IC₅₀ of 18 μM, upon irradiation, produced a related photoproduct structure IC₅₀=9.2 μM. (Nakadaira et al. (1969) J. Chem. Soc. Chem. Commun. 1469) For comparison the IC₅₀ values for ginkgolides A, its 10-keto analog, and the photoproduct of the 10-keto analog (Nakadaira et al. (1969) J. Chem. Soc. Chem. Commun. 1469) were found to be 1.9 μM, 3.9 μM and 0.7 μM, respectively. Similar strategies may be employed to obtain the analogous F-ring eliminated, “simple analogs” of GA and GJ.

In addition Weinges et al. ((1997) Liebigs Ann. Recueil. 1997: 991-3) have described an “F” ring opened form monoacid derivative of GA. Briefly, hydrogenation (in glacial acetic acid with Pt as catalyst) of 3,14-didehydro-10-hydroxyginkgolide (3, 14-anhydroginkgolide A), resulted in the formation of 3,14-didehydro-10-hydroxyginkgolide-15-acid. Further reaction with diazomethane led to the methyl ester, which was transformed into its dimethoxymethane (MOM) derivative form. Ozonolysis of the dimethoxymethane (MOM) derivative form resulted in the formation of a stable secondary ozonide, as well as 10-methoxymethoxy-3-oxo-14,15,16-trinorginkgolide.

Analogous strategies may be employed to obtain the F-ring opened, monoacid and structural analogue forms of GJ.

In addition to ring opened (and deleted) forms of the ginkgolides, a number of reports of the synthesis of ginkgolide derivatives having modifications at position C-7 having the chirality of that designated for both GA derivatives and GJ derivatives, have been described (see Teng, (1991) U.S. Pat. No. 5,241,084; Cazaux et al. (1995) (GB 2288599); Jaracz et al. (2004) Chem.-A Europ. J. 10: 1303 and Jaracz et al. (2002) J. Org. Chem. 67:4623-26). These methods may be applied to obtain certain of the GA and GC derivatives of the invention having a substituted “R₂” group (with either a GA derivative or a GJ derivative chirality) at position C-7).

For example, U.S. Pat. No. 5,241,084 describes a process for preparing GB from GC. This method may be applied to the conversion of GJ to GA. Furthermore, GB 2288599 describes a two-step process for converting GC to GB that involves first reacting ginkgolide C with a sulphonic anyhydride to obtain 7-sulfphonyloxy-ginkgolide B, and, in the second step, reacting the 7-sulfphonyloxy-ginkgolide B with borohydride to eliminate the 7-sulphonyloxy group and thus produce ginkgolide B.

In addition, Vogensen et al. ((2003) J. Med. Chem. 46: 601-8) describe methods for synthesizing a series of ginkgolide derivatives having modifications at the C-7 position. The chirality of the C-7 atom here is described in terms of α and β forms, whereby the β form corresponds to the Ginkgolide J derivative chiral form at C-7 described herein, with R₂ substitutions having the R chiral form, while the a form corresponds to the Ginkgolide A derivative chiral form, as described herein, with R₂ substitutents having the S chiral form. Briefly, various R₂ substitutions at C(7) and in the alpha configuration, corresponding to GA derivatives of the invention, were synthesized by reacting GC with trifluoromethanesulfonic (Tf) anhydride which reacted with remarkable selectivity at the C(7)-OH (higher selective reactivity of C(7)-OH was also observed under strong acidic conditions). The resulting 7β-OTf-GB intermediate was then reacted with various nucleophiles to give the corresponding C-7 substituted derivatives. For example, reaction with sodium acetate yielded 7α-O-acetate-GB; reaction with sodium phenyl acetate yielded 7α-O-phenylacetate-GB; reaction with sodium azide yielded 7α-azido-GB; reaction with tetrabutylammonium fluoride hydrate yielded 7α-fluoro-GB; reaction with tetrabutylammonium chloride yielded 7α-chloro-GB. In addition, a similar chemical approach was used to produce 7α-N-methylamino-GB, 7α-ethylamino-GB, and 7α-ethylamino-GB.

Additional methods for synthesizing GA and GJ derivatives are described in Jaracz et al. ((2004) Chem.-A Europ. J. 10: 1303), which discloses methods to prepare various ether-, ester-, and carbomyl-derivatives of ginkgolides by selective derivitization of the hydroxyl groups, including the C(7)-OH of GJ. Accordingly, these methods may be applied to the synthesis of GJ derivatives having the R chiral form, as defined herein (or the β-configuration at C-7). In brief, the method includes selective alkylation at C(10)-OH with various benzyl-derived halides under mild reaction conditions to yield corresponding C(10)-OR blocked intermediates. Next, the C-7 position is derivatized as desired by esterification in the presence of pyridine.

Still other methods for synthesizing GA and GJ derivatives are described in, Jaracz et al. ((2002) J. Org. Chem. 67:4623-26), which discloses methods for making selective acetylations and other modifications to ginkgolides, including acetylations at C(7)-OH. Their results demonstrated the differences in reactivity among the hydroxyl groups of GC. Acetylation in the presence of base led to higher reactivity of 10-OH, while the pattern for acid-catalyzed acetylation of GC is different as seen in the case of H₂SO₄-catalyzed acetylation reaction. These observations can be explained by the hard-soft and acid-base (HSAB) principle: due to the hydrogen bonding between 1-OH and 10-OH, it is easy to form a delocalized alkoxy anion at 1-OH and 10-OH in the presence of a relatively mild base. This anion becomes a hard base compared to 7-OH. In the presence of acid, the alkoxy anion at 1-OH and 10-OH cannot be formed and 7-OH becomes the harder base, and thus more reactive toward acetylation. Further rational chemical synthetic strategies for synthesizing C(7)-altered ginkgolide derivatives are thereby supported. In addition, Hu et al. ((2000) Bioorg. Medic. Chem. 8: 1515-21) describe methods for synthesizing alkyl and alkoxycarbonyl derivatives of ginkgolide (including those having C(7)-OH that are prepared in one step through alkylation and acylation.

In addition to these methods for chemical synthesis of ginkgolide derivatives, U.S. Pat. No. 6,693,091 describes analogs of terpe trilactones, including ginkgolide derivatives and methods for their synthesis (see also Stromgaard et al. (2002) J Med. Chem. 45: 4038-46). U.S. Pat. No. 5,541,183, describes ginkgolide derivatives, including those having C(10)-OR substitutions and/or C(1)-OR substitutions. Furthermore, U.S. Pat. No. 5,466,829 describes certain ginkgolide derivatives and processes for preparing them. U.S. Pat. No. 6,143,725 describes the synthesis of a number of glycosylated ginkgolide derivatives. U.S. Pat. No. 5,599,950 describes methods for converting GC into GB, which chemical methodologies can be applied to the synthesis of GA and GJ derivatives as well (see also U.S. Pat. No. 5,241,084, described above). As with all the references cited herein, the entire content of each of these cited U.S. Patents are hereby incorporated herein in their entirety.

### 5.6 Antioxidants

The invention is directed to ginkgolide compositions, and methods of using such compositions, that may optionally include antioxidants in their formulation. Antioxidant agents which may be employed in accordance with the above-described methods include vitamin C, vitamin E, N-acetyl-L-cysteine, resveratrol, coenzyme Q, alpha-lipoic acid, lycopene, or any combination thereof. In addition, antioxidant minerals, such as selenium, copper, zinc, manganese, and carotenoids, such as lutein, lycopene, or alpha- and/or beta-carotene may be utilized in the ginkgolide compositions and therapeutics of the invention. In particular, antioxidant agents such as vitamin A, vitamin C, and vitamin E (alpha-tocopherol and other active tocopherols), folic acid, ubiquinone (coenzyme Q10), 2,3-dihydroxy benzoic acid, and 2,5-dihydroxy benzoic acid are useful antioxidants of the invention. Exemplary antioxidants, particularly for oral delivery of the ginkgolide compositions of the invention, are described in U.S. Pat. Nos. 6,551,629 and 6,572,899.

In another aspect, the ginkgolide compositions of the invention are optimized to prevent oxidation during use by including one or more antioxidant compounds. For example, vitamin C (supplied as in the particularly stable form of L-ascorbic acid-2-phosphate or in any other form) may be used. Examples of other antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like. Furthermore N-acetyl-L-cysteine, a potent antioxidant, which is also a precursor of L-cysteine and which thereby promotes the intracellular synthesis of glutathione, may be a particularly useful antioxidant to include in certain cell culture applications (e.g. in vitro applications) of the invention. Other particularly useful antioxidants which may be included in cell culture applications are vitamin E (e.g., D-alpha-tocopherol), resveratrol (a plant phytoalexin derived from, e.g., grapes), coenzyme Q, alpha-lipoic acid, lycopene, bioflavonoids, and quercetin. The form and concentration of each such antioxidant is known in the art or may be discerned with routine testing. For example, L-ascorbic acid-2-phosphate may be used at a concentration of about 20 uM to about 2 mM (e.g., at 0.2 mM), and N-acetyl-L-cysteine may be used at a concentration of about 0.2 to about 20 mM (e.g. at 2 mM).

In certain instances, the antioxidant may be a flavonoid, such as a flavonoid derived from a Ginkgo biloba or other plant extract, as is known in the art. Flavonoids, abundant polyphenols found in plants, are antioxidant molecules having a “flavan nucleus,” and can be classified into ten groups based on differences in their chemical structures (see www.herbalchem.net). These groups include anthocyanidins (e.g., as found in grapes, bilberry blueberries and cocoa), and anthocyanins (e.g. as found in elderberry), proanthocyanidins (e.g., as found in red wine, blueberries, blackberries, and other red/blue/purple colored plant parts), flavanols (e.g., flavan-3-ols) (found in green tea, cocoa powder, red wine, and motherwort), flavonols (e.g., flavonol glycosides found in Ginkgo biloba including quercetin, kaempferol, and isorhamnetin), flavones (e.g., as found in celery and red peppers), and flavanones (e.g., as found in citrus plants), isoflavones (e.g., genistein, as found in red clover, alfalfa, peas, soy and other legumes).

### 5.7 The Extracts of the Invention

The invention also provides extracts comprising one or more terpene trilactones, including but not limited to a ginkgolide, abilobalides or a flavonoid. The extracts can be obtained from natural or unnatural sources.

In one embodiment, an Extract of the Invention is obtained from an unnatural source.

In another embodiment, an Extract of the Invention is obtained from a natural source. Natural sources, include, but are not limited to a plant, a tree, soil, an insect, an animal, a fungus, a mold, a coral, a sponge, or any part thereof. The natural source may be found on land or may exist in an aqueous environment, such as a river, lake, ocean or sea.

In a specific embodiment, the Extract of the Invention is obtained from the Ginkgo Biloba tree.

The Extracts of the Invention may be obtained by extracting a natural or unnatural source using an organic solvent, inorganic solvent, water, or mixtures thereof. The initial extract may be further refined, isolated or purified using methods well known to the skilled artisan to provide Extracts of the Invention which contain one or more terpene trilactones in various amounts.

In one embodiment, an Extract of the Invention comprises from about 10% to about 100% terpene trilactones (by total weight of the extract). In various embodiments an Extract of the Invention comprises from about 20% to about 75% terpene trilactones, from about 30% to about 60% terpene trilactones, or from about 40% to about 50% terpene trilactones.

In one embodiment, an Extract of the Invention comprises greater than 50% terpene trilactones.

In another embodiment, an Extract of the Invention comprises about 55% terpene trilactones.

In yet another embodiment, an Extract of the Invention comprises about 60% terpene trilactones.

In another embodiment, an Extract of the Invention comprises about 65% terpene trilactones.

In still another embodiment, an Extract of the Invention comprises about 70% terpene trilactones.

In a further embodiment, an Extract of the Invention comprises about 75% terpene trilactones.

In another embodiment, an Extract of the Invention comprises about 80% terpene trilactones.

In yet another embodiment, an Extract of the Invention comprises about 85% terpene trilactones.

In another embodiment, an Extract of the Invention comprises about 90% terpene trilactones.

In a further embodiment, an Extract of the Invention comprises about 95% terpene trilactones.

In another embodiment, an Extract of the Invention comprises about 98% terpene trilactones.

The Extracts of the Invention are useful in the present methods for treating or preventing Alzheimer's disease; treating or preventing a neurological or neurodegenerative disease or disorder; treating or preventing a memory disorder; improving memory; treating or preventing depression; protecting a neuron against neuronal cell death and/or long term potentiation impairment caused by beta amyloid protein; and stimulating axonal outgrowth of a neuron.

In one embodiment, the invention provides methods for treating or preventing Alzheimer's disease; treating or preventing a neurological or neurodegenerative disease or disorder; treating or preventing a memory disorder; improving memory, treating or preventing depression; protecting a neuron against neuronal cell death and/or long term potentiation impairment caused by beta amyloid protein; or stimulating axonal outgrowth of a neuron, the methods comprising administering to a subject in need thereof an effective amount of an Extract of the Invention.

In one aspect, the invention provides an extract consisting essentially of ginkgolide A and ginkgolide J, wherein the extract is obtained using a process comprising the steps of:

(i) extracting Ginkgo Biloba plant material with ethyl acetate and filtering the resultant solution to provide a first filtered residue and a first filtrate;

(ii) diluting the first filtered residue with diethyl ether and filtering the resultant solution to provide a second filtered residue and a second filtrate;

(iii) diluting the second filtered residue with methanol and filtering the resultant solution to provide a third residue and a third filtrate;

(iv) concentrating the third filtrate and subjecting the resultant concentrate to chromatograpy under conditions sufficient to provide a first fraction containing a mixture of Ginkgolide A and Ginkgolide B, and a second fraction containing a mixture of Ginkgolide C and Ginkgolide J;

(v) combining the first and second fractions obtained in step (iv) and concentrating the resultant solution to provide a concentrate containing Ginkgolide A, Ginkgolide B, Ginkgolide C and Ginkgolide J;

(vi) diluting the concentrate obtained in step (v) with an organic solvent and contacting the components of the resultant solution with benzyl bromide in the presence of a non-nucleophilic base under conditions sufficient to provide a product mixture containing unreacted Ginkgolide A, unreacted Ginkgolide J, benzylated Ginkgolide B and benzylated Ginkgolide C;

(vii) subjecting the product mixture obtained in step (vii) to chromatography under conditions sufficient to provide a composition comprising Ginkgolide A and Ginkgolide J, wherein the composition does not contain any of: benzylated ginkgolide A, benzylated ginkgolide J, ginkgolide B, benzylated ginkgolide B, ginkgolide C or benzylated ginkgolide C; and

(viii) purifying the composition obtained in step (vii) such that the purified composition consists essentially of Ginkgolide A and Ginkgolide J.

In another aspect, the invention provides an extract comprising more than 10% terpene trilactones, wherein the proportion of terpene trilactones, by weight of the total amount of terpene trilactiones, is from about 52% to about 62% bilobalide, from about 10% to about 20% ginkgolide A, from about 5% to about 15% ginkgolide B, from about 5% to about 15% ginkgolide C, and from about 1% to about 8% ginkgolide J, and wherein the percentages of bilobalide, ginkgolide A, ginkgolide B, ginkgolide C and ginkgolide J add up to 100%.

In one embodiment, the extract comprises about 65% terpene trilactones.

In one embodiment, the extract comprises about 70% terpene trilactones.

In still another aspect, the invention provides an extract comprising more than 10% terpene trilactones, wherein the proportion of terpene trilactones, by weight of the total amount of terpene trilactiones, is from about 20% to about 30% bilobalide, from about 37% to about 47% ginkgolide A, from about 10% to about 20% ginkgolide B, from about 10% to about 20% ginkgolide C, and from about 1% to about 8% ginkgolide J, and wherein the percentages of bilobalide, ginkgolide A, ginkgolide B, ginkgolide C and ginkgolide J present in the composition add up to 100%.

In one embodiment, the extract comprises about 65% terpene trilactones.

In one embodiment, the extract comprises about 70% terpene trilactones.

In one embodiment, the extracts of the invention may further contain one or more of the following compounds:

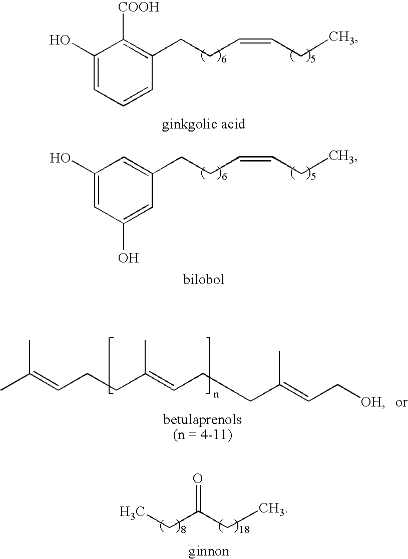

### 5.8 Pharmaceutical Formulations

The ginkgolide compositions of the invention may be administered together with any suitable pharmaceutically acceptable carrier. Furthermore, the ginkgolide compositions of the invention may be formulated for administration in any convenient way (e.g. formulated together with one or more pharmaceutically acceptable carriers (additives) and/or diluents which facilitate any means of administration). The ginkgolide compounds may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam. However, in certain embodiments the subject compounds may be simply dissolved or suspended in sterile water.

The amount of a composition of the invention, which is effective in the treatment or prevention of neurological or neurodegenerative condition can be determined by standard clinical techniques. In addition, in vitro assays may optionally be employed to help identify optimal dosage ranges (see Examples). The precise dose to be employed in the formulation will also depend on the route of administration, and the seriousness of the disease or disorder, and is decided according to the judgment of the practitioner and each patient's circumstances. Effective doses may be extrapolated from dose-response curves derived from in vitro or animal model test systems.

As described above, formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form, will generally be that amount of the compound, which produces a therapeutic effect. Generally, out of one hundred percent, this amount will range from about one percent to about ninety-nine percent of active ingredient, particularly from about 5 percent to about 70 percent, most particularly from about 10 percent to about 30 percent.

The ginkgolide compositions of the invention may be administered together with any suitable pharmaceutically acceptable carrier, such as a pharmaceutically acceptable salt or excipient, e.g., saline or distilled water. In certain instances, diffusion of the compositions of the invention may be facilitated by excipients such as salts, sugars or alcohols. The compound may be optionally administered as a pharmaceutically acceptable salt.

The formulations described herein may optionally include preservatives, such as antioxidants, that stabilize the ginkgolide, and thereby maintaining therapeutic activity. Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Furthermore, excipients such as salts, sugars and alcohols, may optionally be used to facilitate diffusion of the ginkgolide therapeutic. Non-limiting representative excipients that can be used in combination with the present invention include saccharides, such as sucrose, trehalose, lactose, fructose, galactose, mannitol, dextran and glucose; poly alcohols, such as glycerol or sorbitol; proteins, such as albumin; hydrophobic molecules, such as oils; and hydrophilic polymers, such as polyethylene glycol, among others. Pharmaceutical formulations of compounds of the invention described herein includes isomers such as diastereomers and enantiomers, mixtures of isomers, including racemic mixtures, salts, solvates, and polymorphs thereof.

In certain cases, the compounds of the present invention may contain one or more acidic functional groups and, thus, are capable of forming pharmaceutically acceptable salts with pharmaceutically acceptable bases. The term “pharmaceutically-acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as, but not limited to, the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Non-limiting representative organic amines useful for the formation of base addition salts include ethylamine, diethylamine, ethylenediamine, ethanolamine, diethanolamine, piperazine and the like.

The pharmaceutically-acceptable carrier may be a pharmaceutically-acceptable material, composition or vehicle, such as a liquid or solid filler, diluent, excipient, solvent or encapsulating material, involved in carrying or transporting the subject ginkgolide compositions from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include, but are not limited to: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

Therapeutic formulations may be in the form of liquid solutions or suspensions. Methods well known in the art for making formulations are found, for example, in Remington: The Science and Practice of Pharmacy (20th ed., ed. A. R. Gennaro AR.), Lippincott Williams & Wilkins, 2000. For oral administration, formulations may be in the form of tablets or capsules. Intranasal formulations may be in the form of powders, nasal drops, or aerosols. Formulations for parenteral administration may, for example, contain excipients, sterile water, or saline, polyalkylene glycols such as polyethylene glycol, oils of vegetable origin, or hydrogenated napthalenes. Formulations for inhalation may contain excipients, for example, lactose, or may be aqueous solutions containing, for example, polyoxyethylene-9-lauryl ether, glycolate and deoxycholate, or may be oily solutions for administration in the form of nasal drops, or as a gel. The concentration of the compound in the formulation will vary depending upon a number of factors, including the dosage of the drug to be administered, and the route of administration.

The ginkgolide composition may be encapsulated within or administered with a biocompatible polymer to provide controlled release of the ginkgolide compound(s). The biocompatible polymer can be either a biodegradable polymer or a biocompatible non-degradable polymer which releases over time the incorporated ginkgolide composition by diffusion. The ginkgolide composition can be homogeneously or heterogeneously distributed within the biocompatible polymer. A variety of biocompatible polymers are useful in the practice of the invention, the choice of the polymer depending on the rate of ginkgolide composition release required in a particular treatment regimen. The ginkgolide composition can be provided in a polymeric sustained release formulation in which the amount of ginkgolide composition in the composition varies from about 0.1% to about 30%, from about 0.1% to about 10%, or from about 0.5% to about 5% (w/w).

Non-limiting representative synthetic, biodegradable polymers include, for example: polyamides such as poly (amino acids) and poly (peptides); polyesters such as poly (lactic acid), poly (glycolic acid), poly (lactic-co-glycolic acid), and poly (caprolactone); poly (anhydrides); polyorthoesters; polycarbonates; and chemical derivatives thereof (substitutions, additions of chemical groups (e.g., alkyl, alkylene), hydroxylations, oxidations, and other modifications routinely made by those skilled in the art), copolymers and mixtures thereof. The degradable sustained released composition can have a half-life for the release of the ginkgolide composition of greater than one week, two weeks, three weeks, one month, two months, three months, or four months when placed in the body, or otherwise in contact with cells (e.g., neurons) to be treated.

The ginkgolide composition can also be encapsulated within a biocompatible non-degradable polymer. Non-limiting representative non-degradable polymers include polysaccharides; polyethers, such as poly (ethylene oxide), poly (ethylene glycol), and poly (tetramethylene oxide); vinyl polymers, such as polyacrylates, acrylic acids, poly (vinyl alcohol), poly (vinyl pyrolidone), and poly (vinyl acetate); polyurethanes; cellulose-based polymers, such as cellulose, alkyl cellulose, hydroxyalkyl cellulose, cellulose ethers, cellulose esters, nitrocellulose, and cellulose acetates; polysiloxanes and other silicone derivatives.

Useful polymeric sustained released compositions are a solid particulate having an average diameter of less than 400 μm, 200 μm, 100 μm, or 50 μm.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the ginkgolide composition is optionally mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polypropylene oxide copolymer; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate OT cross-linked sodium carboxymethyl cellulose), surface-active or dispersing agent Molded tablets may be made, by molding, in a suitable machine a mixture of the powdered compound moistened with an inert liquid diluent.

The invention also provides kits comprising one or more containers filled with one or more of the ginkgolide compositions of the invention. Optionally associated with such container(s) can be a notice in the form prescribed by a governmental agency regulating the manufacture, use or sale of pharmaceuticals or biological products.

The compounds and compositions of the invention may also be formulated as “neutraceutical” products.

### 5.9 Therapeutic Applications

This invention provides therapeutic methods and compositions useful for treating neurodegeneration and loss of cognitive function. The methods of the invention involve administering to a subject a therapeutic compound which effects neuroprotection and/or cognition enhancement. Neuroprotection and/or cognition enhancement can be effected, for example, by blocking neurotoxicity associated with protein aggregate formation (e.g. amyloid beta plaque formation), or by blocking the inhibition of neuronal long-term portentiation that is associated with such protein aggregate formation.

Many neurodegenerative diseases, including Alzheimer's and Parkinson's and the transmissible spongiform encephalopathies (prion diseases), are characterized by neuronal loss and the accumulation of protein aggregates that are typically fibrillar in structure. Huntington's disease, a hereditary neurodegenerative disease, is characterized by the accumulation of nuclear protein inclusion in the brain Taylor et al. (2002) Science 296: 1991-5; Ross (2002) Neuron 35: 819-22) and huntingtin, the affected protein, has been shown to form amyloid fibrils in vitro (Scherzinger et al. (1997) Cell 90: 549-58). Indeed many neurodegenerative diseases have been found to be associated with the accumulation of apparently neurotoxic protein aggregates including: amyotrophic lateral sclerosis, which is characterized by the intraneuronal inclusions of insoluble superoxide dismutase protein; transmissible spongiform encephalopathies, which are characterized by accumulation of extracellular plaques of protease-resistant prion protein (PrP); fronto-temportal dementia, which is characterized by accumulation of neurofibrillary tangles of fibrillar tau protein; diffuse Lewy body disease, which is characterized by cortical Lewy bodies of fibrillar alpha-synuclein, familial British dementia, which is characterized by extracellular amyloid plaques of fibrillar ABri protein; familial Danish dementia, which is characterized by accumulation of extracellular plaques of insoluble ADan protein; and spinocerebellar ataxia, which is characterized my the appearance of intranuclear neuronal inclusions see Caughey and Lansbury (2003) Annu. Rev. Neurosci. 26: 267-98). Remarkably, the accumulation of neurotoxic protein deposits, such as amyloid beta and tau, even appears to occur in brain injury, such as occurs from traumatic head injury (Franz et al. (2004) Neurology 60: 1457-61; Lambri et al. (2001) Clin. Neuropathol. 20: 263-71). Notably, even Down's syndrome is characterized by the expression of abnormally high levels of the amyloid precursor protein (AAP) and its product the amyloid-beta protein (Lemere et al. (1996) Neurobiol. Dis. 3: 16-32). Therefore protein aggregation appears to be a common widespread cause of neurodegeneration in a multitude of otherwise clinically distinct neurological disease and disorders. Indeed, the ability of such protein aggregation to directly cause neurodegenerative neuronal cell death is supported by a wealth of scientific evidence including: (i) pathology (the colocalization of neuronal cell death and protein aggregates); (ii) genetics (the genes linked to familial forms of these disease encode the aggregated protein); (iii) animal modeling (overexpression of the aggregated proteins produces disease-associated phenotypes (Wong et al. (2002) Nat. Neurosci. 5: 633-39); (iv) biophysics (disease-associated mutations promote in vitro aggregation (Jarret and Lansbury (1993) Cell 75: 1055-58); and (v) mathematical modeling (rates of cell death (Clarke et al. (2000) Nature 406: 195-99) and disease onset and progression are consistent with a nucleation-dependent aggregation process (Perutz and Windle (2001) Nature 412: 143-44; Eigen (1996) Biophys. Chem. 63: A1-18). Accordingly, protein aggregate-induce cell death is a common essential link in neurodegenerative disease.

Significantly, protein aggregates play a role in, not only neurotoxicity, but also the reversible loss of cognitive functions associated with inhibition of neuronal long-term potentiation (LTP) (reviewed in Kourie (2001) Cell. Mol. Neurobiol. 21: 173-213; and Turner et al. (2003) Pros. Neurobiol. 70:1-32). LTP is a form of synaptic information storage that is a well-established central process for memory mechanisms. The inhibition of neuronal LTP by aggregatable proteins such amyloid beta protein, has been associated with reversible memory loss (Gong et al. (2003) PNAS 100: 10417-22). Notably, neuronal LTP inhibition is distinct from neurotoxicity due to protein aggregation. In particular, neuronal LTP effects have a rapid onset without significantly affecting basal synaptic transmission, and there are amyloid beta peptide mutations which uncouple the two effects—i.e, substitutions at βA position 35 block neurotoxicity, but not LTP inhibition (Chen et al. (2000) Neurosci Res. 60: 65-72).

The invention provides ginkgolide compositions that are capable of inhibiting, blocking or reversing both the neurotoxicity resulting from abnormal protein aggregates, and the inhibition of neuronal long-term potentiation associated with such aggregatable proteins. The invention thereby provides compositions and methods for treating or preventing otherwise irreversible neurodegenerative diseases and disorders, as well as for treating or preventing reversible cognition-impairment, such as memory impairment (including age-related memory impairment) and learning disorders.

The invention thereby provides ginkgolide compositions and associated methods for treating patients with a neurological or neurodegenerative disease or disorder such as Alzheimer's disease, multiple sclerosis, amyotrophic lateral sclerosis, Alpers' disease, corticobasal ganglionic degeneration, multiple system atrophy, motor neuron disease, olivopontocerebellar atrophy, Parkinson's disease, prion disease, Rett syndrome, tuberous sclerosis, Shy-Drager syndrome, Huntington's disease, Pick's disease, Creutzfeldt-Jakob disease, depression, aging, head trauma, stroke, CNS hypoxia, cerebral senility, multi-infarct dementia and other neurological conditions including acute neuronal diseases, senile dementia, epileptic dementia, presenile dementia, post-traumatic dementia, vascular dementia and post stroke dementia, alcoholism, meningitis, diabetic peripheral neuropathy, neonatal hypoxia, stroke, global cerebral ischemia, or any combination thereof. Examples of other neurodegenerative conditions subject to therapeutic treatments of the invention include acute neurodegenerative disorders such as stroke, traumatic brain injury, schizophrenia, peripheral nerve damage, hypoglycemia, spinal cord injury, epilepsy, and anoxia and hypoxia. In general, the subject neurodegenerative disorders for treatment include any disorder in which progressive loss of neurons occurs either in the peripheral nervous system or in the central nervous system.

The invention also provides methods of treating neurological conditions and/or preventing an undesirable mental condition (e.g., memory loss) by administering to a subject an effective amount of a therapeutic ginkgolide compound capable of mitigating neurodegeneration. In addition, the subject ginkgolides, and associated methods, provide beneficial neuroprotective effects. Neuroprotection is the inhibition of progressive deterioration of neurons that leads to nerve cell death. Therefore, in the methods of the invention, neurodegeneration in a subject is mitigated, and/or neuroprotection and/or cognition enhancement is effected, by administering a therapeutic compound of the invention to the subject.

In a particularly useful aspect, the invention provides ginkgolide compositions and methods for treating dementias. Dementias are diseases that include memory loss and additional intellectual impairment separate from memory. The invention provides methods for treating patients suffering from memory impairment in all forms of dementia. Dementias are classified according to their cause and include: neurodegenerative dementias (e.g., Alzheimer's, Parkinson's disease, Huntington's disease, Pick's disease), vascular (e.g., infarcts, hemorrhage, cardiac disorders), mixed vascular and Alzheimer's, bacterial meningitis, Creutzfeldt-Jakob Disease, multiple sclerosis, traumatic (e.g., subdural hematoma or traumatic brain injury), infectious (e.g., HIV), genetic (down syndrome), toxic (e.g., heavy metals, alcohol, some medications), metabolic (e.g., vitamin B12 or folate deficiency), CNS hypoxia, Cushing's disease, psychiatric (e.g., depression and schizophrenia), and hydrocephalus. The present invention includes methods for dealing with memory loss separate from dementia, including mild cognitive impairment (MCI) and age-related cognitive decline. The present invention includes methods of treatment for memory impairment as a result of disease. In another application, the invention includes methods for dealing with memory loss resulting from the use of general anesthetics, chemotherapy, radiation treatment, post-surgical trauma, and therapeutic intervention.

In one embodiment, the invention provides methods for treating Alzheimer's disease comprising administering to a subject an effective amount of Ginkgolide J.

In another embodiment, the invention provides methods for treating Alzheimer's disease comprising administering to a subject an effective amount of a composition consisting essentially of Ginkgolide A and Ginkgolide J.

The ginkgolide compositions of the invention are also useful in the treatment of depression. Depression can be the result of organic disease, secondary to stress associated with personal loss, or idiopathic in origin. There is a strong tendency for familial occurrence of some forms of depression suggesting a mechanistic cause for at least some forms of depression. The diagnosis of depression is made primarily by quantification of alterations in patients' mood. These evaluations of mood are generally performed by a physician or quantified by a neuropsychologist using validated rating scales, such as the Hamilton Depression Rating Scale or the Brief Psychiatric Rating Scale. Numerous other scales have been developed to quantify and measure the degree of mood alterations in patients with depression, such as insomnia, difficulty with concentration, lack of energy, feelings of worthlessness, and guilt. The standards for diagnosis of depression as well as all psychiatric diagnoses are collected in the Diagnostic and Statistical Manual of Mental Disorders (Fourth Edition) referred to as the DSM-IV-R manual published by the American Psychiatric Association, 1994.

The ginkgolide compositions of the invention are further useful to improve or enhance memory in a normal subject, e.g., a normal human subject without a neurological disease or disorder, or other form of neurological trauma. In certain applications, the ginkgolide compositions are used to enhance general cognitive function in such a normal human subject. In particular, the compositions of the invention promote neuronal long-term potentiation, a neurocellular function that is central to learning and memory. The ability of these compositions to improve memory may be further be demonstrated in a variety of routine animal tests (see Staubli et al. (1994) Proc. Natl. Acad. Sci. 91: 777-781; and Arai et al. (1996) The Journal of Pharmacology and Experimental Therapeutics 278: 627-638).

Subjects for treatment with the ginkgolide compositions of the invention include living organisms in which the particular neurological condition to be treated can occur. Examples of subjects include humans, apes, monkeys, cows, sheep, goats, dogs, cats, mice, rats, and transgenic species thereof. As would be apparent to a person of skill in the art, the animal subjects employed in the working examples set forth below are reasonable models for human subjects with respect to the tissues and biochemical pathways in question, and consequently the methods, therapeutic compounds and pharmaceutical compositions directed to same. Dosage forms for animals such as, for example, rats can be and are widely used directly to establish dosage levels in therapeutic applications in higher mammals, including humans.

The pharmacological agents of the invention may be advantageously shown to be neuroprotective in one or more animal models known in the art (see e.g., van Leuve (2000) Prog. Neurobiol. 61: 305-12). Agents that are neuroprotective in such animal models may, accordingly, be applied in human therapeutics, after appropriate adjustment of dosage. Specifically, there are comparable memory-deficit patterns between brain-damaged rats and humans, which indicates that the rat can serve as an excellent animal model to evaluate the efficacy of pharmacological treatments or brain damage upon memory.

As would also be apparent to a person skilled in the art, the invention further encompasses methods of the invention employed ex vivo or in vitro. For example, the Examples describe studies utilizing excised neuronal tissue as well as isolated neurons. Furthermore, diagnostic tests or studies of efficacy of selected compounds may conveniently be performed ex vivo or in vitro, including in animal models. Such tests, studies and assays are within the scope of the invention.

The amount of the compound, composition or extract of the invention that is effective to: (1) treat or prevent a neurological or neurodegenerative disease or disorder, a memory disorder; or depression, or (2) protecting a neuron against neuronal cell death, and/or long term potentiation impairment by beta amyloid protein; or to simulate axonal outgrowth of a neuron, can be determined by standard clinical techniques. In addition, in vitro or in vivo assays can optionally be employed to help identify optimal dosage ranges. The precise dose to be employed can also depend on the route of administration, and the seriousness of the condition being treated and can be decided according to the judgment of the practitioner and each subject's circumstances in view of, e.g. published clinical studies. Suitable effective dosage amounts, however, range from about 10 micrograms to about 5 grams about every 4 h, although they are typically about 500 mg or less per every 4 hours. In one embodiment the effective dosage is about 0.01 mg, 0.5 mg, about 1 mg, about 50 mg, about 100 mg, about 200 mg, about 300 mg, about 400 mg, about 500 mg, about 600 mg, about 700 mg, about 800 mg, about 900 mg, about 1 g, about 1.2 g, about 1.4 g, about 1.6 g, about 1.8 g, about 2.0 g, about 2.2 g, about 2.4 g, about 2.6 g, about 2.8 g, about 3.0 g, about 3.2 g, about 3.4 g, about 3.6 g, about 3.8 g, about 4.0 g, about 4.2 g, about 4.4 g, about 4.6 g, about 4.8 g, and about 5.0 g, every 4 hours. Equivalent dosages can be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The effective dosage amounts described herein refer to total amounts administered; that is, if more than one compound, composition or extract of the invention is administered, the effective dosage amounts correspond to the total amount administered.

In one embodiment, the effective dose for a compound, composition or extract of the invention of the invention is from about 0.1 mg/kg to about 1000 mg/kg of a subject's body weight per day. In various embodiments, the effective dose for a compound, composition or extract of the invention of the invention is from about 0.5 mg/kg to about 50 mg/kg, and from about 1 mg/kg to about 10 mg/kg.

Compositions can be prepared according to conventional mixing, granulating or coating methods, respectively, and the present compositions can contain, in one embodiment, from about 0.1% to about 99%; and in another embodiment from about 1% to about 70% of the active component(s) by weight or volume.

The dosage regimen utilizing the compound, composition or extract of the invention can be selected in accordance with a variety of factors including type, species, age, weight, sex and medical condition of the subject; the severity of the condition to be treated; the route of administration; the renal or hepatic function of the subject; and the particular compound, composition or extract of the invention employed. A person skilled in the art can readily determine the effective amount of the drug useful for: (1) treating or preventing a neurological or neurodegenerative disease or disorder; a memory disorder; or depression, or (2) protecting a neuron against neuronal cell death, and/or long term potentiation impairment by beta amyloid protein; or to simulate axonal outgrowth of a neuron.

The compounds, compositions or extracts of the invention can be administered in a single daily dose, or the total daily dosage can be administered in divided doses of two, three or four times daily. Furthermore, the compounds, compositions or extracts of the invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration can be continuous rather than intermittent throughout the dosage regimen. Other illustrative topical preparations include creams, ointments, lotions, aerosol sprays and gels, wherein the concentration of compound, composition or extract of the invention ranges from about 0.1% to about 15%, w/w or w/v.

The compound, composition or extract of the invention can be assayed in vitro or in vivo for the desired therapeutic or prophylactic activity prior to use in humans. Animal model systems can be used to demonstrate safety and efficacy.

The present methods useful for: (1) treating or preventing a neurological or neurodegenerative disease or disorder; a memory disorder; or depression, or (2) protecting a neuron against neuronal cell death, and/or long term potentiation impairment by beta amyloid protein; or to simulate axonal outgrowth of a neuron, in a subject in need thereof can further comprise administering another prophylactic or therapeutic agent to the subject being administered a compound, composition or extract of the invention. In one embodiment the other prophylactic or therapeutic agent is administered in an effective amount.

The other prophylactic or therapeutic agent includes, but is not limited to, a cholinesterase inhibitor, a phosphodiesterase inhibitor, an N-methyl D-aspartate antagonist, acetyl-L-carnitine, phosphatidylserine, melatonin, vitamin B6, vitamin B12, vitamin C, vitamin E, or any agent known to be useful in the treatment of Alzheimer's disease, dementia, or any disease or condition having an adverse effect on cognitive function.

In one embodiment, the other therapeutic agent is a phosphodiesterase inhibitor. In one embodiment, the phosphodiesterase inhibitor is an inhibitor of phosophodiesterase IV. In another embodiment, the phosphodiesterase inhibitor is an inhibitor of phosophodiesterase V. In another embodiment, the phosphodiesterase inhibitor is an inhibitor of phosophodiesterase X. In a specific embodiment, the phosphodiesterase IV inhibitor is rolipram.

In one embodiment, the other therapeutic agent is a cholinesterase inhibitor. Cholinesterase inhibitors useful in the methods of the present invention include, but are not limited to, tacrine, galantapine, donezepil and rivastigmine.

In another embodiment, the other therapeutic agent is a N-methyl D-aspartate antagonist. N-methyl D-aspartate antagonists useful in the methods of the present invention include, include but are not limited to, memantine.

In one embodiment, the other therapeutic agent is memantine.

In another embodiment, the other therapeutic agent is donezepil.

In one embodiment, the compound, composition or extract of the invention can be administered prior to, concurrently with, or after another prophylactic or therapeutic agent, or on the same day, or within 1 hour, 2 hours, 12 hours, 24 hours, 48 hours or 72 hours of each other.

Effective amounts of the other prophylactic or therapeutic agents are well known to those skilled in the art. However, it is well within the skilled artisan's purview to determine the other prophylactic or therapeutic agent's optimal effective amount range. In one embodiment of the invention, where another prophylactic or therapeutic agent is administered to a subject, the effective amount of the compound, composition or extract of the invention is less than its effective amount would be where the other prophylactic or therapeutic agent is not administered. In this case, without being bound by theory, it is believed that the compounds, compositions or extracts of the invention and the other prophylactic or therapeutic agent act synergistically to (1) treat or prevent a neurological or neurodegenerative disease or disorder; a memory disorder; or depression, or (2) protecting a neuron against neuronal cell death, and/or long term potentiation impairment by beta amyloid protein; or to simulate axonal outgrowth of a neuron.

The compounds, compositions and extracts of the invention can be effective when administered orally. Accordingly, in one embodiment, the route of administration is oral administration. Alternatively, the active compound may be administered by other suitable routes such as transdermal, subcutaneous, intraocular, intravenous, intramuscular or intraperitoneal administration, and the like (e.g., by injection). Depending on the route of administration, the active compound may be coated in a material to protect the compound from the action of acids, enzymes and other natural conditions which may inactivate the compound.

The compounds of the invention can be formulated to ensure proper distribution in vivo. For example, the blood-brain barrier (BBB) excludes certain highly hydrophilic compounds. In certain embodiments, the invention includes ginkgolide compositions containing hydrophilic flavonoid compounds. To ensure that certain of the compounds, compositions and extracts of the invention cross the BBB, they can be formulated, for example, in liposomes. For methods of manufacturing liposomes, see, e.g. U.S. Pat. Nos. 4,522,811; 5,374,548; and 5,399,331. The liposomes may comprise one or more moieties which are selectively transported into specific cells or organs (“targeting moieties”), thus providing targeted drug delivery. Exemplary targeting moieties include folate and biotin (see, e.g., U.S. Pat. No. 5,416,016); antibodies; and surfactant protein A receptor. Accordingly, in certain instances, the compounds, compositions and extracts of the invention are formulated in liposomes; and in particular instances, the liposomes include a targeting moiety.

Delivery and in vivo distribution can also be affected by alteration of a substituent group of a compound of the invention.

To administer the therapeutic compound by other than parenteral administration, it may be necessary to coat the compound with, or co-administer the compound with, a material to prevent its inactivation. For example, the therapeutic compound may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al., 1984). The therapeutic compound may also be administered parenterally (e.g., intramuscularly, intravenously, intraperitoneally, intraspinally, or intracerebrally). Dispersions can be prepared in glycerol, liquid polyethylene glycols, and mixtures thereof and in oils. Under ordinary conditions of storage and use, these preparations may contain a preservative to prevent the growth of microorganisms. Pharmaceutical compositions suitable for injectable use include sterile aqueous solutions (where water soluble) or dispersions and sterile powders for the extemporaneous preparation of sterile injectable solutions or dispersions. In all cases, the composition must be sterile and must be fluid to the extent that easy syringability exists. It must be stable under the conditions of manufacture and storage and must be preserved against the contaminating action of microorganisms such as bacteria and fungi. The vehicle can be a solvent or dispersion medium containing, for example, water, ethanol, polyol (for example, glycerol, propylene glycol, liquid polyethylene glycol, and the like), suitable mixtures thereof, and vegetable oils. The proper fluidity can be maintained, for example, by the use of a coating such as lecithin, by the maintenance of the required particle size in the case of dispersion, and by the use of surfactants.

Prevention of the action of microorganisms can be achieved by the addition of various antibacterial and antifungal agents, for example, parabens, chlorobutanol, phenol, ascorbic acid, thimerosal, and the like. In some cases, it may be advantageous to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic compound in the required amount in an appropriate solvent with one or a combination of ingredients enumerated above, as required, followed by filter sterilization. Generally, dispersions are prepared by incorporating the therapeutic compound into a sterile vehicle which contains a basic dispersion medium and the required other ingredients from those enumerated above. In the case of sterile powders for the preparation of sterile injectable solutions, the methods of preparation include vacuum drying and freeze-drying which yield a powder of the active ingredient (i.e., the therapeutic compound) optionally plus any additional desired ingredient from a previously sterile-filtered solution thereof.

The therapeutic compound can be orally administered, for example, with an inert diluent or an assimilable edible carrier. The therapeutic compound and other ingredients may also be enclosed in a hard or soft shell gelatin capsule, compressed into tablets, or incorporated directly into the subject's diet. For oral therapeutic administration, the therapeutic compound may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The a specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of neurological conditions in subjects.

Therapeutic compositions can be administered in time-release or depot form, to obtain sustained release of the therapeutic compounds over time. The compounds, compositions and extracts of the invention can also be administered transdermally (e.g. by providing the therapeutic compound, with a suitable carrier, in patch form).

Active compounds are administered at a therapeutically effective dosage sufficient to mitigate neurodegeneration and/or to effect neuroprotection and/or cognition enhancement in a subject. In various embodiments, a “therapeutically effective dosage” mitigates neurodegeneration by about 20%, by about 40%, by about 60%, and by about 80% relative to untreated subjects. The ability of a compound to mitigate neurodegeneration can be evaluated in model systems that may be predictive of efficacy in mitigating neurodegeneration in human diseases, such as animal model systems known in the art (including, e.g., the method of transient middle cerebral artery occlusion in the rat) or by in vitro methods, (including, e.g., the assays described herein).

It will be appreciated that the ability of a compound of the invention to mitigate neurodegeneration will, in certain embodiments, be evaluated by observation of one or more symptoms or signs associated with neurodegeneration in vivo. Thus, for example, the ability of a compound to mitigate neurodegeneration may be associated with an observable improvement in a clinical manifestation of the underlying neurodegeneration-related disease state or condition, or a slowing or delay in progression of symptoms of the condition. Thus, monitoring of clinical manifestations of disease can be useful in evaluating the neurodegeneration-mitigating efficacy of a compound of the invention.

The compounds and compositions of the invention may be administered as nutritional supplements as neutraceutical products.

### 5.10 Compositions and Methods for Imaging

The invention provides compositions, and methods of use, of Ginkgolide A and Ginkgolide J, and derivatives thereof, for imaging (e.g., diagnostic biomedical imaging in a human patient). U.S. Pat. No. 6,693,091 describes the synthesis and use of analogs of the terpene trilactones from Ginkgo biloba for bioorganic and imaging studies.

In certain applications, one or more atoms of the GA, GJ or a derivative of GA or GJ may be labeled, e.g. by incorporation of a suitable radiolabel during chemical synthesis or by bioincorporation in a Ginkgo biloba plant. In other applications, a labeled, detectable by one or more imaging methods, may be incorporated at the “R₂” group of Ginkgolide A or J. Suitable R₂ labels include photoactivatable moieties, fluorescent moieties, and radioactive moieties.

The invention thereby provides a process for detecting the binding of Ginkgolide A or Ginkgolide J, or a derivative thereof, to a target, by contacting the compound with the target and detecting the binding of the compound to the target. The target may be, for example, an enzyme or a receptor, particularly, one which plays a role in neuronal cell survival and/or neuronal long-term potentiation processes.

This invention further provides a process for detecting the localization of a receptor in a subject by administering any of the described compounds to the subject and detecting at any location in the subject's body to identify a point of accumulation of the compound so as to thereby localize the receptor in the subject. Localization of a receptor means a higher concentration of that receptor then at other points in the subject's body. For example, particular receptors, so localized, may be those active hi neuronal long-term potentiation and cell survival, and, more particularly, in the reversal of neuronal long-term potentiation resulting from beta-amyloid protein.

For example, the photoactivatable moieties may react with a receptor, enzyme or other target upon irradiation and enable researchers to identify the targets of compounds, to determine the affinity and selectivity of the drug-target interaction, and to identify the binding site on the target Examples are presented from three fundamentally different approaches: (1) photoaffinity labeling of target macromolecules; (2) photoactivation and release of “caged ligands”; and (3) photoimmobilization of ligands onto surfaces. A number of photoactivatable moieties are described in the literature, for example, aryl azides, which, when photoactivated to yield aryl nitrenes, can label any binding site containing carbon-hydrogen bonds by insertion into the C—H bond (Galardy, et al., J. Biol. Chem., 249: 350 (1974); U.S. Pat. No. 4,689,310; and U.S. Pat. No. 4,716,122); and a number of others are described in U.S. Pat. No. 6,077,698, the contents of which are hereby incorporated by reference.

The photoactivatable groups can be used for treatment as well as screening studies and diagnostics. Photoactivatable groups can be used to irreversibly bind compounds to their targets. Thus, the subject invention also provides compounds useful in methods of treatment where a desired compound is irreversibly bound to its target.

The photoactivatable groups may be phenylazides, purine and pyrimidine azides, 8-azidoadenosine, 2-azidoadenosine, diazoacetates, diazoketones, nitrobenzenes, aryldiazonium salts, or 3H,3-aryldiazirines. Photoactivatable moieties useful in the present invention include, but are not limited to benzophenone, trifluoromethyldiazirine and tetrafluorophenyl.

The fluorescent moiety, for example, may be 5-(dimemethylamino)naphthalene-sulfonyl chloride (dansyl chloride), a fluorescent amine such as 1-pyrenemethylamine, or any number of other groups readily available from Molecular Probes—http://www.probes.com/, the contents of which are hereby incorporated by reference. Other specific groups which are useful in this invention are 1-(Bromoacetyl)pyrene, 3-Bromoacetyl-7-diethylaminocoumarin, 3-Bromomethyl-6,7-dimethoxycoumarin, 8-Bromomethyl-4,4-difluoro-1,3,5,7-tetramethyl-4-bora-3a,4a-diaza-s-indace ne, 3-Bromomethyl-6,7-dimethoxy-1-methyl-2(¹H)-quinoxazolinone, 6-Bromoacetyl-2-dimethylaminonaphthalene, and 4-(9-Anthroyloxy)phenacyl bromide.

Radioactive moieties are widely known in the art and include radionuclides, radionuclides covalently attached to other groups, and metal chelates. The appropriate ginkgolide-based radioligands can be prepared using known radioactive moieties to suit the environment of use and detection method. Gamma-emitter and positron-emitter radionuclides are well-known in the art and include ¹¹¹In, ¹⁹⁸Au, ¹¹³Ag, ¹¹¹Ag, ¹²³I, ¹²⁵I, ¹³⁰I, ¹³¹I, ¹³³I, ¹³⁵I, ⁴⁷SC, ⁷²As, ⁷²Se, ⁹⁰Y, ⁸⁸Y, ⁹⁷Ru, ¹⁰⁰Pd, ¹⁰⁹Pd, ¹⁰⁵Rh, ¹²⁸Ba, ¹⁹⁷Hg, ²⁰³Pb, ²¹²Pb, ⁶⁷Ga, ⁶⁸Ga, ⁶⁴Cu, ⁶⁷Cr, ⁹⁷Ru, ⁷⁵Br, ⁷⁶Br, ⁷⁷Br, ^{99m}Tc, ¹¹C, ¹³N, ¹⁵O, ³H and ¹⁸F.

Radionuclides useful in the present methods relating to positron emission tomography (PET) studies include, but are not limited to ¹⁸F, ¹¹C and ³H.

The following examples are set forth to assist in understanding the invention and should not, of course, be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### 6. EXAMPLES

Certain aspects of the invention are supported and further described in particular detail in the following illustrative examples.

### 6.1 Example 1

### Preparation of β-amyloid 1-42

In these experiments the toxic activity of the oligomeric form of β-amyloid 1-42 is examined. Among the different aggregation species of the peptide, β-amyloid 1-42 oligomers inhibit synaptic activity and induce cell death at lower concentrations than the fibrillar forms (Dahlgren et al. (2002) J Biol. Chem. 277:32046-53; Stine et al. (2003) J. Biol. Chem. 278:11612-22). Briefly, the lyophilized peptide (American Peptide, Sunnyvale, Calif.) is re-suspended in 1,1,1,3,3,3-Hexafluoro-2-Propanol (HFIP; Sigma-Aldrich, St. Louis, Mo.) to 1 mM using a glass gas-tight Hamilton syringe and divided in aliquots. The solution is then allowed to evaporate in the fume hood. The resulting clear peptide film is further dried under vacuum (6.7 mTorr) in a SpeedVac™ (Savant Instruments; Holbrook, N.Y.). The dessicated pellet can be stored at −20° C. for several months. Immediately prior to use, the aliquots are resuspended to 5 mM in anhydrous dimethylsulfoxide (DMSO) by pipette mixing followed by bath sonication for 10 minutes. In order to obtain Aβ1-42 oligomers, 5 mM Aβ1-42 is diluted in DMSO to 1100M in ice-cold culture medium, immediately followed by vortexing for 30 seconds and incubation at 4° C. for 24 hours.

### 6.2 Example 2

### Terpene Trilactones Reverse Beta Amyloid-Induced Inhibition of Long Term Potentiation

To determine whether terpene trilactones can rescue the inhibition of long-term potentiation (LTP) that is induced by beta-amyloid (1-42) protein, a terpene trilactone 70% enriched preparation (P8A) was used at a concentration of 200 ug/L in a hippocampal LTP assay system as described below.

To prepare hippocampal slices for LTP measurements, mice were decapitated, and their hippocampi were removed. Transverse hippocampal slices of a thickness of 400 μm were maintained in an interface chamber at 29° C. They were perfused with saline solution (124.0 mM NaCL, 4.4 mM KCL, 1.0 mM Na₂HPO₄, 25.0 mM NaHCO₃, 2.0 mM CaCl₂, 2.0 mM MgSO₄, 10 mM glucose) continuously bubbled with 95% O₂ and 5% CO₂. Slices were permitted to recover for at least 90 minutes before recordings. Further details of hippocampal slice preparation, as well as LTP measurement, have been described (see Son et al. (1998) Learn. Mem. 5: 231-45).

To make electrophysiological recording, a concentric bipolar platinum-iridium stimulation electrode and a low-resistance glass recording microelectrode filled with saline solution (5 milliohms resistance) were used. The extracellular field excitatory postsynaptic potential (fEPSP) measurements were recorded from the CA1 region of the hippocampus by placing both the stimulating and the recording electrodes in CA1 stratum radiatum. BST was assayed by plotting the stimulus voltage (V) against slopes of fEPSP to generate input-output relations. For LTP experiments, baseline stimulation was delivered every minute at an intensity that evoked a response approximately 35% of the maximum evoked response. Baseline response was recorded for 15 min. prior to beginning the experiment to assure stability of the response. LTP was induced using theta-burst stimulation (4 pulses at 100 Hz, with the bursts repeated at 5 Hz and each tetanus including 3 ten-burst trains separated by 15 seconds). Drugs or vehicle in 0.1% DMSO were added to the bath solution for 20 min. prior to the induction of LTP. In the LTP blocking experiments, beta-amyloid protein was used to block tetanus-induced LTP by treating acute hippocampal slices with 200 nM oligomeric beta-amyloid protein (βA) (amyloid-beta peptide 1-42 (DAEFRHDSGY EVHHQKLVFF AEDVGSNKGA IIGLMVGGVV IA)) (SEQ ID NO: 1) for 30 minutes, with or without the addition of terpene trilactone or individual ginkgolide or bilobalide preparations.

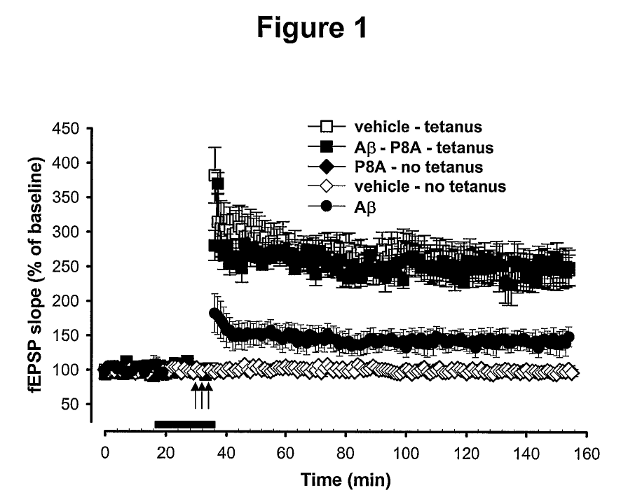

The effect of the enriched terpene trilactone preparation (P8A) on beta-amyloid inhibition of tetanus-induced LTP in these hippocampal neuron preparations are shown in FIG. 1. The horizontal bar indicates the period during which Aβ (200 nM) and P8A (200 μg/L) were added to the bath solution. The arrows indicate the time at which the theta-burst stimulation was applied in this and the following figure. Notably, in this experiment, both beta-amyloid protein (Aβ) and terpene trilactone preparation (P8A) did not affect baseline transmission. The results show that amyloid-beta protein (βA) inhibited tetanus-induced LTP (black circles), but co-application of the terpene trilactone preparation (P8A) reversed the inhibition of LTP by amyloid-beta protein (βA) (black squares), while application of P8A alone had no effect on the baseline transmission (black diamonds).

The results support the ability of one or more ginkgolide terpene trilactone compositions to block the inhibition of long-term potentiation that is associated with beta-amyloid protein and that occurs in neurodegenerative and cognitive-impairing diseases and disorders (see section 4.6).

### 6.3 Example 3

### GA and GJ Alone Reverse Beta Amyloid-Induced Inhibition of Long Term Potentiation

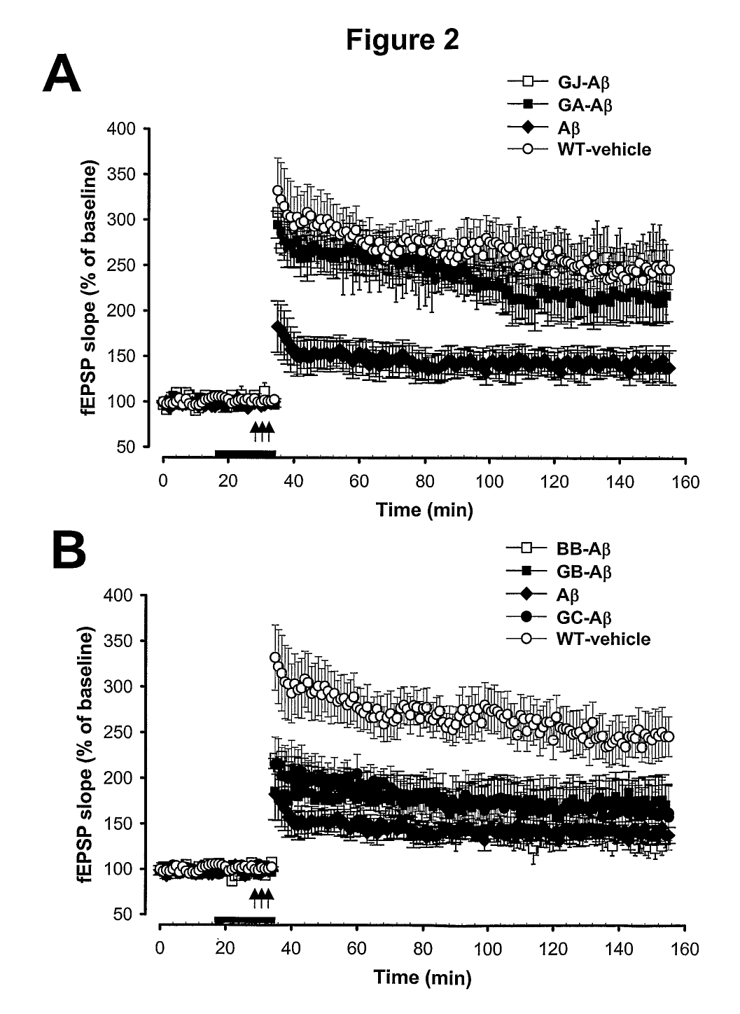

To determine which, if any, of the characterized components of the terpene trilactone preparation (P8A) mediate this neuroprotective effect, the effect of each of the four ginkgolides (GA, GB, GC, and GJ), as well as bilobalide (BB), was tested individually at a concentration of 1 uM. The results show that Ginkgolide A and Ginkgolide J consistently rescued the LTP to the same values recorded in control untreated slices (FIG. 2A), while Ginkgolides B and C and bilobalide did not (FIG. 2B). In detail, FIG. 2A is a summary graph showing that a 20 min. treatment with GJ and GA rescues L-LTP impairment in slices treated with Aβ for 20 min. prior to L-LTP induction. The horizontal bar indicates the period during which these drugs were added to the bath solution. FIG. 2B is a summary graph showing that 20 min. treatment with the ginkgolides GB and GC as well as the bilobalide BB does not rescue L-LTP impairment in slices treated with Aβ for 20 min. prior to L-LTP induction. The horizontal bar indicates the period during which these drugs were added to the bath solution. Experiments in A and B were interleaved with each other. Therefore the components of the P8A terpene trilactone active in this neuroprotective effect were Ginkgolides A and J alone.

The results demonstrate the ability of Ginkgolide A and Ginkgolide J compositions to block the inhibition of long-term potentiation that is associated with beta-amyloid protein and that occurs in neurodegenerative and cognitive-impairing diseases and disorders (see section 5.8).

### 6.4 Example 4

### GJ Alone Protects Against Beta Amyloid-Induced Neuronal Cell Death

To determine whether any of the ginkgolides or bilobalide are able to protect neurons against neurodegenerative cell death, rat hippocampal cultures kept 5-6 days in vitro where exposed for 24 hours to 5-10 μM oligomeric beta amyloid 1-42 with or without the addition of the terpende trilactone preparation (P8A), as well as with each of the ginkgolides or bilobalide at 1 μM.

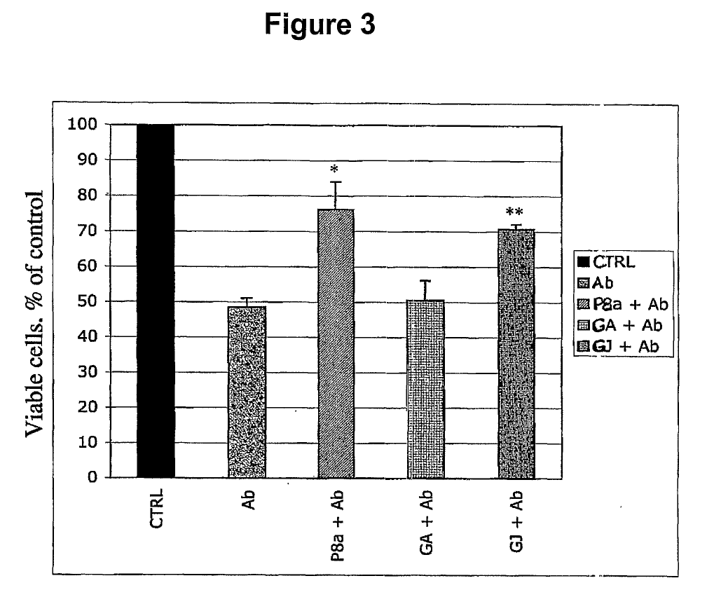

The results are shown in FIG. 3. Hippocampal neuron cultures were prepared according to the method previously described (Vitolo et al. (2002) PNAS 99: 13217-21). Briefly, fetuses at embryonic day 18 (E18) from timed pregnant Sprague-Dawley rats (Taconic Farms) were sacrificed and the hippocampi removed. Neurons were then dissociated, plated on 24 well-plates coated with poly-L-lysine and maintained in a defined serum free medium. The obtained cultures resulted in a population enriched in large pyramidal neurons that constitute one of the primary targets of Alzheimer's disease. After 5-6 days in vitro cells were used for the experiments.

Cultures were treated adding 10 mM beta amyloid 142 in its oligomeric form with or without the 70% enriched fraction of terpene trilactones (terpene trilactones) at 50 μg/ml, or alternatively each one of the ginkgolides (GA, GJ) at a concentration of 1 μM. After 24 h the number of viable cells was assessed using cell nuclei counting. Amyloid beta induced death of 50% of the cell population. Both the terpene trilactones (P8a) and GJ, but not GA or any of the other ginkgolides were consistently able to prevent cell death (Student-Newman-Keuls multiple comparison test, p<0.01 for Ab vs P8a+Ab, *, p<0.05 for Ab vs GJ+Ab, **). In a preliminary experiment, a higher-concentration of GJ (5 μM) appeared to completely prevent amyloid beta toxicity. All the substances did not affect neuronal viability when added alone. CTRL=Control, 100%. Ab=Amyloid beta, 48.5%±2.4. P8a+Ab=terpene trilactones+Amyloid beta, 76.0±7.9. GA+Ab=Ginkgolide A+Amyloid beta, 50.5±5.7, GJ+Ab=Ginkgolide J+Amyloid beta, 70.7±1.4. Values represent mean±SEM of 3 consecutives experiments and each experiment was performed in triplicate. Ginkgolide J consistently protected the hippocampal cultures, which showed approximately 40% more cells than the other treatments.

The results support the ability of Ginkgolide J to prevent aggregatable protein-induced (e.g., βA-induced) neuronal cell death that is associated with a multitude of neurodegenerative diseases and disorders (see section 5.8).

### 6.5 Example 5

### Rapid Vesicle Cycling Assay

In this example, the cationic styrylpiridinium dye FM 1-43, is used to identify actively firing neurons and provide a facile means to assess the effect of new ginkgolide compositions of the invention on neuronal function. The cationic styrylpiridinium dye FM 1-43 is a non-toxic, water-soluble dye that is non-fluorescent when in aqueous solution. Upon binding to the external leaflet of the cell membrane, however, it emits an intense fluorescent light. FM 1-43 is added to the medium of neuronal cultures, and it is rapidly internalized within recycling synaptic vesicles and stains pre-synaptic nerve terminals. Briefly, the cultured neurons are then stimulated by high K⁺ concentration, resulting in the release of neurotransmitter/s and the dye contained in the synaptic vesicle. As a consequence, the intensity of fluorescence in the nerve terminals decreases. The rate of exocytosis is measured as a decrease in fluorescence upon continued synaptic stimulation in dye-free medium.

The loading of FM 1-43 is induced by changing the incubation medium form physiological saline solution (119 mM NaCl, 2.5 mM KCl, 2 mM CaCl₂, 2 mM MgCl₂, 25 mM HEPES and 30 mM glucose) to hyperkalemic incubation solution (31.5 mM NaCl, 90 mM KCl, 2 mM CaCl₂, 2 mM MgCl₂, 25 mM HEPES and 30 mM glucose) with 5 μM FM 1-43 for 45 seconds. The incubation solution is then changed back to physiological solution for 10 minutes to wash off the dye from the medium. In order to enhance the removal of FM 1-43 from the medium, the anionic cyclodextrin complexing agent ADVASEP-7 (1 mM, CyDex, Inc., Overland Park, Kans.) is added after 1 and 6 minutes of washing for 60 seconds. After a 10 minutes wash, which is enough to allow complete recycling and re-priming of the dye-stained population of synaptic vesicles, a fluorescent microscopic photo is taken to record the amount of fluorescence present in the nerve terminals. The cultures are then repeatedly exposed for 15 seconds to the hyperkalemic solution without the FM 1-43 to elicit cycles of exocytosis of the dye in the extracellular medium. After 30 minutes another fluorescent microscopic photo is taken, and neuronal activity is measured as the difference in fluorescence intensity before and after K⁺ stimulation.

Beta-amyloid is added to this fluorescent assay system in the form, and in the concentrations, described above. The added beta-amyloid protein inhibits vesicle cycling. The vesicle cycling correlates with neuronal long-term potentiation function. Derivatives of GA, and/or GJ, as well as other structural analogs of GA and/or GJ, are rapidly screened for their ability to reverse beta-amyloid inhibition of vesicle cycling using this assay. Those with this activity are used in treating neurodegenerative diseases or disorders as well as for the enhancement of cognitive function. Accordingly, this method allows the screening of multiple samples at the same time and, insomuch as it is not a substitute for electrophysiological recordings, it provides a fast and efficient tool to identify drugs, which affect neuron function.

### 6.6 Example 6

### Determination of pCREB Levels

pCREB Determination

Levels of pCREB can be measured after a 15-minute exposure to 50 mM glutamate of hippocampal cultures that have been pre-incubated with amyloid β-peptide (Aβ) either alone or Aβ with 3 mM of a compound, composition or extract of the invention. After the pre-incubation step the culture medium is replaced by a defined salt medium containing 119 mM NaCl, 5 mM KCl, 2 mM CaCl₂ 20 mM HEPES, 1 mM glycine, 300 mM glucose, with 50 mM glutamate (Aβ and the compound, composition or extract of the invention are freshly added to the culture medium at this point to maintain their concentration throughout the experiments). The osmolarity of the medium is then adjusted to about 325 mOsm using sucrose and the pH is raised to about 7.3 using 10 N NaOH. After 15 minutes cells are harvested in 1× modified RIPA with 80 mM glycerophosphate and whole cell extracts are prepared. The lysates are centrifuged at 10,000 rpm for 10 minutes at 4° C. Protein is separated on 12% PAGE and the immunoblotting is performed as described below. Phosphorylated CREB (pCREB) can be detected using an antibody for CREB phosphorylated at Ser133 (1:1000 dilution, Upstate Biotechnology).

1. A compound having the formula: 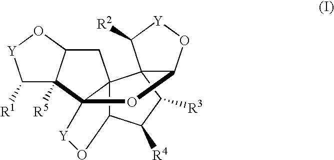
or a pharmaceutically acceptable salt thereof,
wherein:
each occurrence of Y is independently —CH₂— or —C(O)—;
R¹ and R³ are each independently —H or —C₁-C₆ alkyl;
R² is —H, —OH, —O—C₁-C₅ alkyl, —O—C₂-C₅ alkenyl, —O—C₂-C₅ alkynyl, —O—C(O)—C₁-C₅ alkyl, —O—C(O)-aryl, —O—CO—NH—C₁-C₅ alkyl, —O—SO₂—C₁-C₅ alkyl, or —O—SO₂-aryl;
R⁴ is —C₁-C₅ alkyl, —NH₂, -halo, —C₂-C₅ alkenyl, —C₂-C₅ alkynyl, —O—C₁-C₅ alkyl, —O—C₂-C₅ alkenyl, —O—C₂-C₅ alkynyl, —O—C(O)—C₁-C₅ alkyl, —O—C(O)-aryl, —O—CO—NH—C₁-C₅ alkyl, —O—SO₂—C₁-C₅ alkyl, or —O—SO₂-aryl; and
R⁵ is —H or —OH.

2. A composition consisting essentially of two or more structurally distinct compounds, each having the formula: 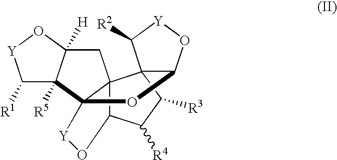
wherein:
each occurrence of Y is independently —CH₂— or —C(O)—;
R¹ and R³ and are each independently —H or —C₁-C₆ alkyl;
R¹ and R³ are each independently —H or —C₁-C₆ alkyl;
R² is —H, —OH, —O—C₁-C₅ alkyl, —O—C₂-C₅ alkenyl, —O—C₂-C₅ alkynyl, —O—C(O)—C₁-C₅ alkyl, —O—C(O)-aryl, —O—CO—NH—C₁-C₅ alkyl, —O—SO₂—C₁-C₅ alkyl, or —O—SO₂-aryl;
R⁴ is —C₁-C₅ alkyl, —NH₂, -halo, —C₂-C₅ alkenyl, —C₂-C₅ alkynyl, —O—C₁-C₅ alkyl, —O—C₂-C₅ alkenyl, —O—C₂-C₅ alkynyl, —O—C(O)—C₁-C₅ alkyl, —O—C(O)-aryl, —O—CO—NH—C₁-C₅ alkyl, —O—SO₂—C₁-C₅ alkyl, or —O—SO₂-aryl; and
R⁵ is —H or —OH.

3. The composition of claim 2, wherein for at least one compound of formula (II), each occurrence of Y is —CH₂—.

4. The composition of claim 2, wherein for at least one compound of formula (II), each occurrence of Y is —C(O)—.

5. The composition of claim 2, wherein for at least one compound of formula (II), R¹ is —C₁-C₆ alkyl.

6. The composition of claim 5, wherein R¹ is methyl.

7. The composition of claim 2, wherein for at least one compound of formula (II), R³ is —C₁-C₆ alkyl.

8. The composition of claim 7, wherein R³ is tert-butyl.

9. The composition of claim 2, wherein for at least one compound of formula (II), R⁴ is —OH.

10. The composition of claim 2, wherein for at least one compound of formula (II), R⁵ is —OH.

11. The composition of claim 2, wherein for at least one compound of formula (II), R² is —OH.

12. A composition consisting essentially of a first compound having the formula: 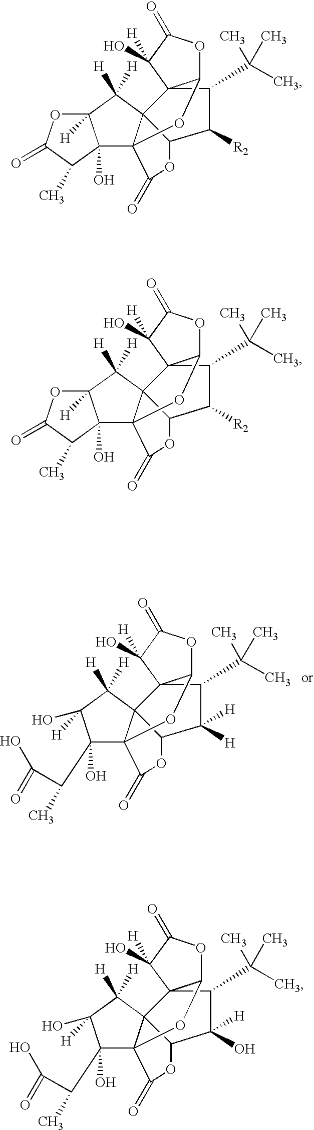
wherein R₂ is —H, —C₁-C₅ alkyl, —OH, —NH₂, -halo, —C₂-C₅ alkenyl, —C₂-C₅ alkynyl, —O—C₁-C₅ alkyl, —O—C₂-C₅ alkenyl, —O—C₂-C₅ alkynyl, —O—C(O)—C₁-C₅ alkyl, —O—C(O)-aryl, —O—CO—NH—C₁-C₅ alkyl, —O—SO₂—C₁-C₅ alkyl, or —O—SO₂-aryl;
and one or more structurally distinct compounds, each having the formula: 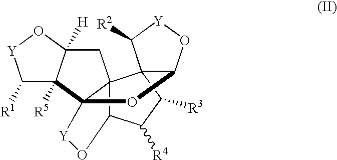
wherein each occurrence of Y is independently —CH₂— or —C(O)—;
R¹ and R³ and are each independently —H or —C₁-C₆ alkyl;
R¹ and R³ are each independently —H or —C₁-C₆ alkyl;
R² is —H, —OH, —O—C₁-C₅ alkyl, —O—C₂-C₅ alkenyl, —O—C₂-C₅ alkynyl, —O—C(O)—C₁-C₅ alkyl, —O—C(O)-aryl, —O—CO—NH—C₁-C₅ alkyl, —O—SO₂—C₁-C₅ alkyl, or —O—SO₂-aryl;
R⁴ is —C₁-C₅ alkyl, —NH₂, -halo, —C₂-C₅ alkenyl, —C₂-C₅ alkynyl, —O—C₁-C₅ alkyl, —O—C₂-C₅ alkenyl, —O—C₂-C₅ alkynyl, —O—C(O)—C₁-C₅ alkyl, —O—C(O)-aryl, —O—CO—NH—C₁-C₅ alkyl, —O—SO₂—C₁-C₅ alkyl, or —O—SO₂-aryl; and
R⁵ is —H or —OH.

13. (canceled)

14. The composition of claim 12, wherein the first compound is a compound having the formula:

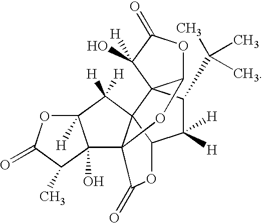

15. The composition of claim 12, wherein the first compound is a compound having the formula:

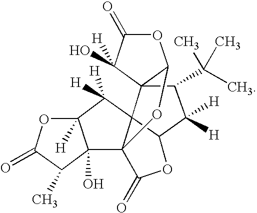

16-17. (canceled)

18. A composition consisting essentially of Ginkgolide A and Ginkgolide J.

19. A composition consisting essentially of (i) an antioxidant, a pharmaceutical carrier, or a combination thereof and (ii) two or more structurally distinct compounds, each having the formula: 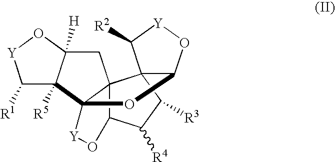
wherein:
each occurrence of Y is independently —CH₂— or —C(O)—;
R¹ and R³ and are each independently —H or —C₁-C₆ alkyl;
R¹ and R³ are each independently —H or —C₁-C₆ alkyl;
R² is —H, —OH, —O—C₁-C₅ alkyl, —O—C₂-C₅ alkenyl, —O—C₂-C₅ alkynyl, —O—C(O)—C₁-C₅ alkyl, —O—C(O)-aryl, —O—CO—NH—C₁-C₅ alkyl, —O—SO₂—C₁-C₅ alkyl, or —O—SO₂-aryl;
R⁴ is —C₁-C₅ alkyl, —NH₂, -halo, —C₂-C₅ alkenyl, —C₂-C₅ alkynyl, —O—C₁-C₅ alkyl, —O—C₂-C₅ alkenyl, —O—C₂-C₅ alkynyl, —O—C(O)—C₁-C₅ alkyl, —O—C(O)-aryl, —O—CO—NH—C₁-C₅ alkyl, —O—SO₂—C₁-C₅ alkyl, or —O—SO₂-aryl; and
R⁵ is —H or —OH.

20. The composition of claim 19, wherein the antioxidant is vitamin C, vitamin E, N-acetyl-L-cysteine, resveratrol, coenzyme Q, alpha-lipoic acid, lycopene, or any combination thereof.

21. The composition of claim 19, wherein the antioxidant is a biflavone.

22. The composition of claim 21, wherein the biflavone is amentoflavone, ailobetin, ginkgetin, isoginlgetin, sciadopirysin, or any combination thereof.

23. The composition of claim 19, wherein the antioxidant is a flavonoid.

24. The composition of claim 23, wherein the flavonoid is a flavonol glycoside.

25. The composition of claim 24, wherein the flavonol glycoside is quercetin, kaempferol, isorhamnetin, or any combination thereof.

26-28. (canceled)

29. The composition of claim 18, wherein the composition is obtained using a process comprising:
(i) extracting Ginkgo Biloba plant material with ethyl acetate and filtering the resultant solution to provide a first filtered residue and a first filtrate;
(ii) diluting the first filtered residue with diethyl ether and filtering the resultant solution to provide a second filtered residue and a second filtrate;
(iii) diluting the second filtered residue with methanol and filtering the resultant solution to provide a third residue and a third filtrate;
(iv) concentrating the third filtrate and subjecting the resultant concentrate to chromatography under conditions sufficient to provide a first fraction which comprises a mixture of Ginkgolide A and Ginkgolide B, and a second fraction which comprises a mixture of Ginkgolide C and Ginkgolide J;
(v) combining the first and second fractions and concentrating the combined first and second fractions to provide a concentrate which comprises Ginkgolide A, Ginkgolide B, Ginkgolide C and Ginkgolide J;
(vi) diluting the concentrate obtained in step (v) with an organic solvent and contacting the components of the resultant solution with benzyl bromide in the presence of a non-nucleophilic base under conditions sufficient to provide a product mixture which comprises unreacted Ginkgolide A, unreacted Ginkgolide J, benzylated Ginkgolide B and benzylated Ginkgolide C;
(vii) subjecting the product mixture obtained in step (vii) to chromatography under conditions sufficient to provide a composition comprising Ginkgolide A and Ginkgolide J; and
(viii) purifying the composition obtained in step (vii) to obtain a purified composition consisting essentially of Ginkgolide A and Ginkgolide J.

30. An extract comprising more than 10% terpene trilactones, wherein the proportion of terpene trilactones, by weight of the total amount of terpene trilactiones, is
from about 20% to about 30% or about 52% to about 62% bilobalide,
from about 10% to about 20% or about 37% to about 47% ginkgolide A,
from about 5% to about 20% ginkgolide B,
from about 5% to about 20% ginkgolide C, and
from about 1% to about 8% ginkgolide J, and
wherein the percentages of bilobalide, ginkgolide A, ginkgolide B, ginkgolide C and ginkgolide J add up to 100%.

31. (canceled)

32. The extract of claim 30, comprising about 65% terpene trilactones.

33. The extract of claim 30, comprising about 70% terpene trilactones.

34. A method of treating a disease or disorder comprising administering to a subject an effective amount of
(i) an enriched Ginkgo biloba extract comprising at least about 60% terpene trilactones; or
(ii) a compound having the formula: 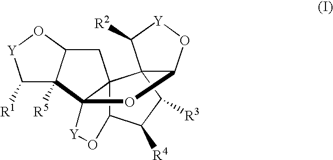
or a pharmaceutically acceptable salt thereof; or
(iii) a composition consisting essentially of two or more structurally distinct compounds, each having the formula: 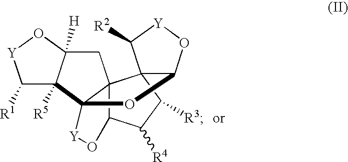
(iv) a composition consisting essentially of a first compound having the formula: 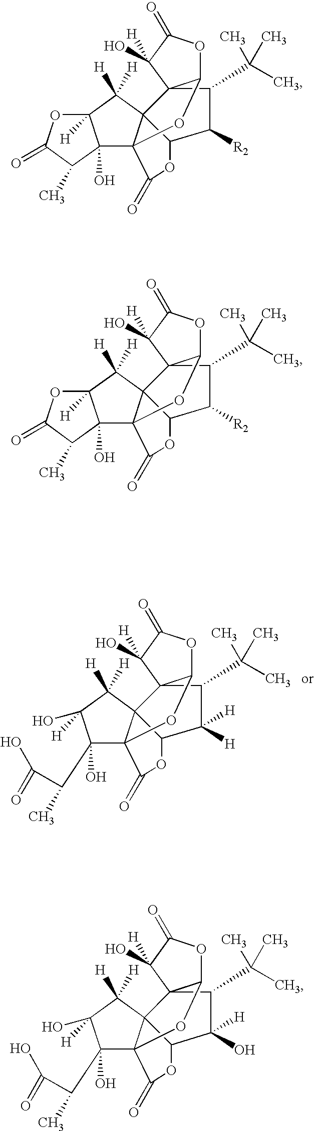
wherein R₂ is —H, —C₁-C₅ alkyl, —OH, —NH₂, -halo, —C₂-C₅ alkenyl, —C₂-C₅ alkynyl, —O—C₁-C₅ alkyl, —O—C₂-C₅ alkenyl, —O—C₂-C₅ alkynyl, —O—C(O)—C₁-C₅ alkyl, —O—C(O)-aryl, —O—CO—NH—C₁-C₅ alkyl, —O—SO₂—C₁-C₅ alkyl, or —O—SO₂-aryl;
and one or more structurally distinct compounds, each having the formula: 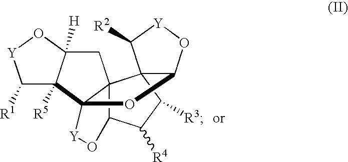
(v) a composition consisting essentially of Ginkgolide A, Ginkgolide J, or combinations thereof; or
(vi) a composition consisting essentially of (a) an antioxidant, a pharmaceutical carrier, or a combination thereof and (b) two or more structurally distinct compounds, each having the formula: 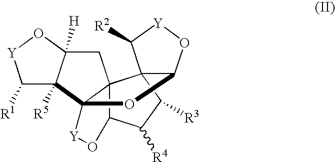
wherein in formula (II):
each occurrence of Y is independently —CH₂— or —C(O)—;
R¹ and R³ and are each independently —H or —C₁-C₆ alkyl;
R¹ and R³ are each independently —H or —C₁-C₆ alkyl;
R² is —H, —OH, —O—C₁-C₅ alkyl, —O—C₂-C₅ alkenyl, —O—C₂-C₅ alkynyl, —O—C(O)—C₁-C₅ alkyl, —O—C(O)-aryl, —O—CO—NH—C₁-C₅ alkyl, —O—SO₂—C₁-C₅ alkyl, or —O—SO₂-aryl;
R⁴ is —C₁-C₅ alkyl, —NH₂, -halo, —C₂-C₅ alkenyl, —C₂-C₅ alkynyl, —O—C₁-C₅ alkyl, —O—C₂-C₅ alkenyl, —O—C₂-C₅ alkynyl, —O—C(O)—C₁-C₅ alkyl, —O—C(O)-aryl, —O—CO—NH—C₁-C₅ alkyl, —O—SO₂—C₁-C₅ alkyl or —O—SO₂-aryl; and
R⁵ is —H or —OH.

35. The method of claim 34, wherein the disease or the disorder is a neurological or neurodegenerative disease or disorder, a memory disorder, depression, neurological damage, Alzheimer's diseases, multiple sclerosis, amyotrophic lateral sclerosis, Alpers' disease, corticobasal ganglionic degeneration, multiple system atrophy, motor neuron disease, olivopontocerebellar atrophy, Parkinson's disease, prion disease, Rett syndrome, tuberous sclerosis, Shy-Drager syndrome, Huntington's disease, senile dementia, epileptic dementia, presenile dementia, post-traumatic dementia, vascular dementia and post stroke dementia, alcoholism, meningitis, neonatal hypoxia, stroke, global cerebral ischemia, or any combination thereof.

36-39. (canceled)

40. A method for protecting a neuron against neuronal cell death or long term potentiation impairment by beta amyloid protein comprising contacting the neuron with
(i) an enriched Ginkgo biloba extract comprising at least about 60% terpene trilactones; or
(ii) a compound having the formula: 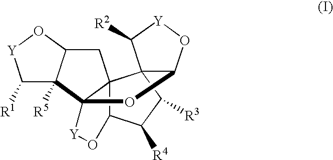
or a pharmaceutically acceptable salt thereof; or
(iii) a composition consisting essentially of two or more structurally distinct compounds, each having the formula: 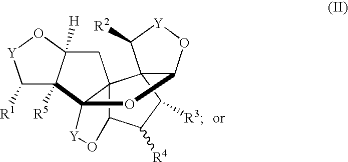
(iv) a composition consisting essentially of a first compound having the formula: 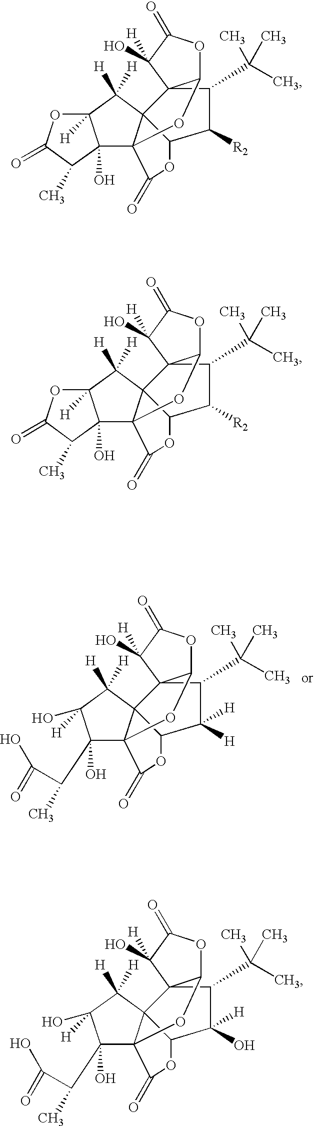
wherein R₂ is —H, —C₁-C₅ alkyl, —OH, —NH₂, -halo, —C₂-C₅ alkenyl, —C₂-C₅ alkynyl, —O—C₁-C₅ alkyl, —O—C₂-C₅ alkenyl, —O—C₂-C₅ alkynyl, —O—C(O)—C₁-C₅ alkyl, —O—C(O)-aryl, —O—CO—NH—C₁-C₅ alkyl, —O—SO₂—C₁-C₅ alkyl, or —O—SO₂-aryl;
and one or more structurally distinct compounds, each having the formula: 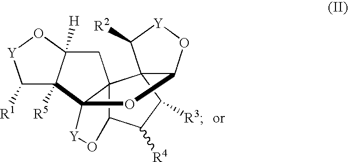
(v) a composition consisting essentially of Ginkgolide A, Ginkgolide J, or combinations thereof; or
(vi) a composition consisting essentially of (a) an antioxidant, a pharmaceutical carrier, or a combination thereof and (b) two or more structurally distinct compounds, each having the formula: 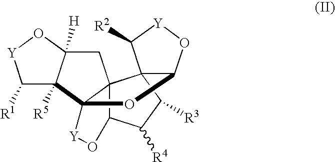
wherein in formula (II):
each occurrence of Y is independently —CH₂— or —C(O)—;
R¹ and R³ and are each independently —H or —C₁-C₆ alkyl;
R¹ and R³ are each independently —H or —C₁-C₆ alkyl;
R² is —H, —OH, —O—C₁-C₅ alkyl, —O—C₂-C₅ alkenyl, —O—C₂-C₅ alkynyl, —O—C(O)—C₁-C₅ alkyl, —O—C(O)-aryl, —O—CO—NH—C₁-C₅ alkyl, —O—SO₂—C₁-C₅ alkyl, or —O—SO₂-aryl;
R⁴ is —C₁-C₅ alkyl, —NH₂, -halo, —C₂-C₅ alkenyl, —C₂-C₅ alkynyl, —O—C₁-C₅ alkyl, —O—C₂-C₅ alkenyl, —O—C₂-C₅ alkynyl, —O—C(O)—C₁-C₅ alkyl, —O—C(O)-aryl, —O—CO—NH—C₁-C₅ alkyl, —O—SO₂—C₁-C₅ alkyl, or —O—SO₂-aryl; and
R⁵ is —H or —OH.

41. A method for stimulating axonal out growth of a neuron comprising contacting the neuron with
(i) an enriched Ginkgo biloba extract comprising at least about 60% terpene trilactones; or
(ii) a compound having the formula: 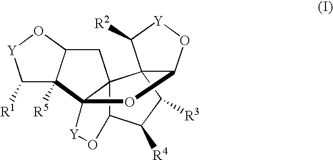
or a pharmaceutically acceptable salt thereof; or
(iii) a composition consisting essentially of two or more structurally distinct compounds, each having the formula: 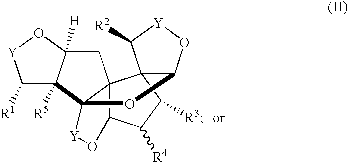
(iv) a composition consisting essentially of a first compound having the formula: 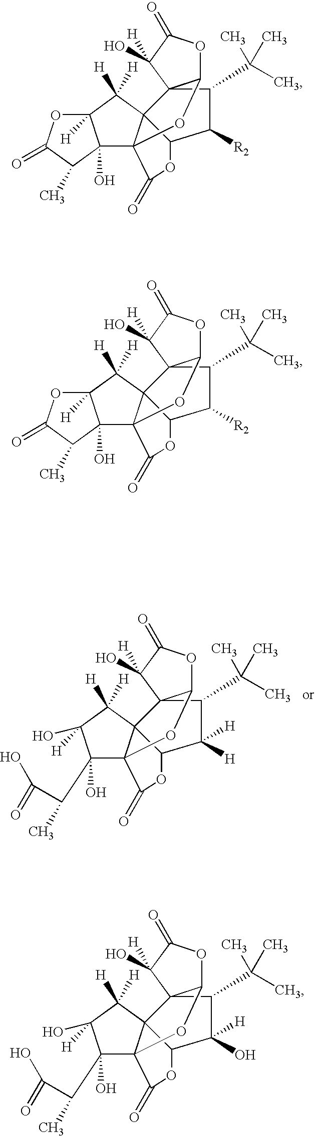
wherein R₂ is —H, —C₁-C₅ alkyl, —OH, —NH₂, -halo, —C₂-C₅ alkenyl, —C₂-C₅ alkynyl, —O—C₁-C₅ alkyl, —O—C₂-C₅ alkenyl, —O—C₂-C₅ alkynyl, —O—C(O)—C₁-C₅ alkyl, —O—C(O)-aryl, —O—CO—NH—C₁-C₅ alkyl, —O—SO₂—C₁-C₅ alkyl, or —O—SO₂-aryl;
and one or more structurally distinct compounds, each having the formula: 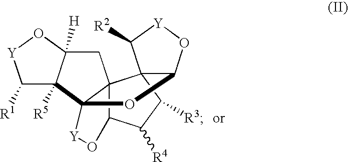
(v) a composition consisting essentially of Ginkgolide A, Ginkgolide J, or combinations thereof; or
(vi) a composition consisting essentially of (a) an antioxidant, a pharmaceutical carrier, or a combination thereof and (b) two or more structurally distinct compounds, each having the formula: 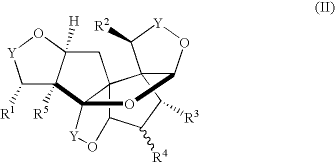
wherein in formula (II):
each occurrence of Y is independently —CH₂— or —C(O)—;
R¹ and R³ and are each independently —H or —C₁-C₆ alkyl;
R¹ and R³ are each independently —H or —C₁-C₆ alkyl;
R² is —H, —OH, —O—C₁-C₅ alkyl, —O—C₂-C₅ alkenyl, —O—C₂-C₅ alkynyl, —O—C(O)—C₁-C₅ alkyl, —O—C(O)-aryl, —O—CO—NH—C₁-C₅ alkyl, —O—SO₂—C₁-C₅ alkyl, or —O—SO₂-aryl;
R⁴ is —C₁-C₅ alkyl, —NH₂, -halo, —C₂-C₅ alkenyl, —C₂-C₅ alkynyl, —O—C₁-C₅ alkyl, O—C₁-C₅ alkenyl, —O—C₂-C₅ alkynyl, —O—C(O)—C₁-C₅ alkyl, —O—C(O)-aryl, —O—CO—NH—C₁-C₅ alkyl, —O—SO₂—C₁-C₅ alkyl, or —O—SO₂-aryl; and
R⁵ is —H or —OH.

42-43. (canceled)

44. A method of identifying a receptor that binds a compound that protects against neuronal cell death or long term potentiation impairment by beta amyloid protein comprising administering the phosphodiesterase inhibitor to a subject a compound having the formula: 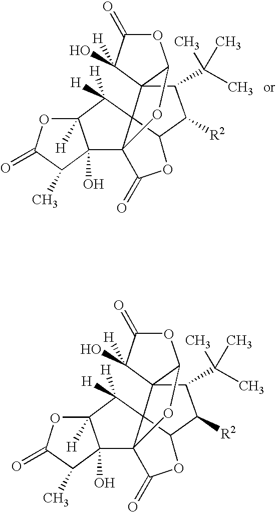
wherein R₂ is a detectable moiety.

45. (canceled)

46. The method of claim 44, wherein the detectable moiety comprises a photoactivatable moiety, a fluorescent moiety, or a radioactive moiety.

47-57. (canceled)


SEQUENCE LISTING (1 sequence)

SEQ ID NO: 1 - protein, 42 aa, Homo sapiens [sequence omitted]